(19) United States
(12) Patent Application Publication
(10) Pub. No.: US 2020/0069582 A1
(43) Pub. Date: Mar. 5, 2020

(54) INHALABLE HEMODYNAMIC AGENTS AND METHODS OF USING THE SAME

(71) Applicant: Darren Rubin, Largo, FL (US)

(72) Inventor: Darren Rubin, Largo, FL (US)

(21) Appl. No.: 16/678,466

(22) Filed: Nov. 8, 2019

Related U.S. Application Data
(63) Continuation-in-part of application No. 16/578,064, filed on Sep. 20, 2019.
Application No. 16/578,064 is a continuation-in-part of application No. 16/130,325, filed on Sep. 13, 2018.
Application No. 16/130,325 is a continuation-in-part of application No. 15/818,056, filed on Nov. 20, 2017.
Application No. 15/818,056 is a continuation-in-part of application No. 14/323,561, filed on Jul. 3, 2014, now abandoned.

(51) Int. Cl. A61K 9/00; A61K 31/137; A61K 9/12; A61M 15/00

(52) U.S. Cl. CPC A61K 9/0073; A61K 31/137; A61M 15/0065; A61K 9/0043; A61K 9/12; A61K 9/0019

(57) ABSTRACT

The present disclosure provides methods of mimicking epinephrine plasma pharmacokinetic parameters/plasma epinephrine levels of an at least one l-epinephrine injection in humans with an at least one intranasal dosage of an inhalable l-epinephrine formulation adapted for transmucosal absorption. Dosages for sublingual or buccal absorption and/or oral inhalation are also provided. These methods may be helpful when l-epinephrine injection is not available or not possible. The present disclosure also provides methods of pretreating breathing difficulty before intranasal treatment of anaphylaxis. The present disclosure allows formulations for intranasal administration of l-epinephrine to be conveniently carried by soldiers and others, such as in a remote location or battlefield, and such as when emergency medical services are not readily available or accessible. The methods may be able to sustain life and restore proper blood perfusion when someone is having cardiopulmonary difficulty until medical help or transport can arrive.

### CROSS REFERENCE TO RELATED APPLICATIONS

The present application is a continuation-in-part of pending U.S. patent application Ser. No. 16/578,064 filed Sep. 20, 2019, which is a continuation-in-part of pending U.S. patent application Ser. No. 16/130,325 filed Sep. 13, 2018, which is a continuation-in-part of U.S. patent application Ser. No. 15/818,056 filed Nov. 20, 2017, which is a continuation-in-part of U.S. patent application Ser. No. 14/323,561 filed Jul. 3, 2014, the subject matter of which applications is incorporated herein by reference in its entirety for all purposes.

### FIELD

The present disclosure relates to methods of mimicking epinephrine plasma pharmacokinetic parameters/plasma epinephrine levels of an at least one l-epinephrine injection in humans with an at least one dosage of a sublingual, intranasal, and/or oral l-epinephrine formulation; and includes methods of maintaining constant elevated plasma epinephrine level(s) by the consecutive dosing of sublingual, intranasal, and/or oral l-epinephrine.

### BACKGROUND

Adequate blood pressure provides the necessary perfusion of blood to tissues so that they receive oxygen and nutrients essential to their metabolic needs. A prolonged drop in blood pressure reduces blood flow and oxygen to these tissues. Eventually, this can lead to the state of circulatory shock: whole body circulatory failure by which tissues and/organs lose perfusion, leading to irreparable, ischemic cellular injury, multiple organ failure, and death. Without immediate treatment, the chances of survival are greatly diminished, and cardiac arrest and respiratory arrest can ensue.

The main components of blood pressure include cardiac output, in terms of heart rate and stroke volume, which is related to heart size and strength of ventricular contraction; along with vascular resistance to flow, which is a function of peripheral vessel vasoconstriction.

Excessive peripheral vasodilation and vessel leakiness occur in conditions like systemic inflammatory response syndrome (SIRS), and in anaphylaxis and sepsis, which lead to anaphylactic shock and septic shock. These types of shock are associated with a decreased systemic vascular resistance and are distributive in nature: a subset of circulatory shock. With anaphylaxis, a severe allergic reaction, such as to a food or insect bite, triggers massive histamine release by mast cells, resulting in vasodilation. With sepsis, a blood infection, endotoxins released from blood borne pathogens, such as gram-negative bacteria, lead to low perfusion by damaging endothelial cells of the vessels to release vasodilators, such as nitric oxide. Endotoxins also activate the complement pathway to release histamine from mast cells, another vasodilator. Endotoxins also activate macrophages and neutrophils of the immune system that release cytokines for a pro-inflammatory cascade that further damages endothelial cells, leading to increased vascular permeability or leakiness of the blood vessels that can decrease intravascular fluid volume. Both conditions are associated with a significant drop in blood pressure.

Hemodynamics can at least temporarily be restored by the emergency administration of Epinephrine Injection, the chemically synthesized drug product form of this endogenous catecholamine hormone, otherwise known as adrenaline, produced by the adrenal gland. Epinephrine acts on both alpha- and beta-adrenergic receptors. The mechanism of the rise in blood pressure is from epinephrine's three-fold actions of direct myocardial stimulation that increases the strength of ventricular contraction (positive inotropic action), an increased heart rate (positive chronotropic action), and peripheral vasoconstriction. Its action on alpha-adrenergic receptors is what lessens both the vasodilation and increased vascular permeability, which occur during anaphylaxis and sepsis.

Epinephrine Injection has a rapid onset and short duration of action. In a hospital setting, Epinephrine Injection is administered intravenously, e.g., by continuous intravenous infusion for treating hypotension associated with septic shock. But staying alive and getting to an emergency room, or having emergency medical services arrive at the scene, is not always possible in a timely manner. For individuals susceptible to allergic reactions, such as certain asthmatics and those with peanut allergy, autoinjectors of Epinephrine Injection are often prescribed and carried around for intramuscular (IM) or subcutaneous injection.

Epinephrine autoinjectors are not without issue. Firstly, they are large and bulky to carry around, especially because a second unit may be needed. So some patients may not always have the autoinjector with them. Then there are issues with storing them out of the heat (e.g., a hot glove compartment may degrade the product) and replacing them often (e.g., annually) due to a short shelf-life. Because of their high cost, some families forgo this expense altogether at great risk, while some use product well beyond its expiration date. Confusion about instructions of use with proper technique can also be an issue. If a patient triggers the device prematurely, the device becomes useless, and the dose does not get administered. Even when a patient has an epinephrine autoinjector, many are reluctant to use it because of needle fear. No doubt these autoinjectors hurt badly. The long needle is under great tension by a strong spring, and pushes outward with tremendous force. If an adult administers an autoinjector to a struggling child, cases of large cuts or gashes along the leg have been reported. And with the obesity epidemic, many experts believe autoinjector needles may not be long enough to provide intramuscular injection to all patients.

There is a tremendous need for a safer alternative, as well as a more convenient and universal method of administering epinephrine to restore hemodynamics in an emergency situation, such as for anaphylaxis, sepsis, anaphylactic shock, septic shock, or even respiratory difficulty and cardiac difficulty, to sustain life and restore proper blood perfusion when someone is having cardiopulmonary difficulty until medical help or transport can arrive. If epinephrine was available in tablet form, many of the above issues and limitations of epinephrine autoinjectors would be eliminated. In tablet form, several doses of epinephrine can easily be carried and associated with a much longer shelf-life than autoinjectors, and fear of needles would not deter its use. The problem is that epinephrine is not conducive to oral administration. Epinephrine quickly degrades in the stomach/intestines and liver, and therefore, has poor oral bioavailability when ingested, and may cause stomach pain or discomfort if swallowed.

The term “transmucosal drug delivery” is drug delivery through a mucous membrane. Mucous membranes, or the mucosae, comprise the linings of bodily cavities and are of mostly endodermal origin and covered in epithelium, which are involved in absorption and secretion. Most absorption by the body takes place at the intestinal mucosa after oral, or enteral, administration of food and/oral medications. Enteral means “through the intestines,” and may also include through the rectal mucosa by enema or suppository. But other mucosa are able to absorb medications, including the oral and buccal mucosa lining the mouth, the bronchial mucosa inside the lungs, and the nasal mucosa inside the nose.

Although non-intestinal mucosal administration routes represent a potential opportunity, in regards to sublingual administration of epinephrine powder or epinephrine, orally disintegrating tablets or films can be swallowed accidentally, or saliva mixing with the disintegrated or dissolved product can be swallowed. This would result in loss of epinephrine due to the degradation of epinephrine associated with the oral route and first-pass metabolism by enzymes of the gastrointestinal tract and liver.

### SUMMARY

Methods of elevating plasma epinephrine levels are disclosed herein. Some methods include providing a first dosage and a second dosage of an inhalable l-epinephrine formulation to a patient by dispensing at least one of the dosages from a dispensing container into or past the patient's nostril(s) for nasal inhalation or intranasal delivery. The inhalable l-epinephrine formulation is adapted for transmucosal absorption. At least one of blood pressure, pulse, and breathing is maintained or substantially stabilized or normalized in the patient.

Some methods include providing at least two dosages of an inhalable l-epinephrine formulation to a patient. One of the two dosages is greater than the other dosage and is dispensed from a dispensing container into or past the patient's nostril(s) for nasal inhalation or intranasal delivery. At least one of blood pressure, pulse, and breathing is maintained or substantially stabilized or normalized in the patient.

Some methods include providing a dosage of an inhalable bronchodilator formulation to a patient by dispensing the dosage from a dispensing container into or past the patient's mouth for delivery to the patient's lungs by oral inhalation. A dosage of an inhalable l-epinephrine formulation is provided to the patient by dispensing the dosage from a dispensing container into or past the patient's nostril(s) for nasal inhalation or intranasal delivery. The inhalable l-epinephrine formulation is adapted for transmucosal absorption. At least one of blood pressure, pulse, and breathing is maintained or substantially stabilized or normalized in the patient.

### BRIEF DESCRIPTION OF THE DRAWINGS

The present disclosure will be better understood and objects other than those set forth above will become apparent when consideration is given to the following detailed description thereof. Such description makes reference to the annexed drawings demonstrating embodiments of the disclosure wherein:

FIG. 1 shows in linear scale, the mean baseline corrected epinephrine plasma concentrations (pg/mL) plotted against nominal time (hours) for human subjects dosed with ADRENATAB™ l-epinephrine sublingual tablets made with the Tartrate Salt form of epinephrine active ingredient compared to human subjects dosed with a 0.3 mg intramuscular epinephrine injection into the lateral part of the thigh.

FIG. 2 shows in logarithmic scale, the mean baseline corrected epinephrine plasma concentrations (pg/mL) plotted against nominal time (hours) for human subjects dosed with ADRENATAB l-epinephrine sublingual tablets made with the Tartrate Salt form of epinephrine active ingredient compared to human subjects dosed with a 0.3 mg intramuscular epinephrine injection into the lateral part of the thigh.

FIG. 3 shows in linear scale, the mean baseline corrected epinephrine plasma concentrations (pg/mL) plotted against nominal time (hours) for human subjects dosed with ADRENATAB l-epinephrine sublingual tablets made with the Base form of epinephrine active ingredient compared to human subjects dosed with a 0.3 mg intramuscular epinephrine injection into the lateral part of the thigh.

FIG. 4 shows in logarithmic scale, the mean baseline corrected epinephrine plasma concentrations (pg/mL) plotted against nominal time (hours) for human subjects dosed with ADRENATAB l-epinephrine sublingual tablets made with the Base form of epinephrine active ingredient compared to human subjects dosed with a 0.3 mg intramuscular epinephrine injection into the lateral part of the thigh.

### DETAILED DESCRIPTION

The present disclosure is directed to formulations of l-epinephrine for sublingual administration, intranasal administration, or oral inhalation, and methods of using any or all. The terms “person,” “subject,” “user,” “patient,” and the like are used interchangeably herein and are not meant to limit the mode of administration or use.

### Sublingual Epinephrine

In some aspects, this disclosure relates to delivering l-epinephrine via the non-intestinal mucosae, such as in the mouth, including sublingual, sublabial, buccal, palate, and gingiva sites. Swallowing epinephrine may result in its rapid degradation in the stomach and metabolization in intestinal mucosa and the liver. The present disclosure includes methods that help avoid the swallowing of epinephrine when administered, such as by transmucosal routes inside the mouth. These methods also apply to other hemodynamic agents to raise blood pressure.

The present disclosure provides methods of l-epinephrine sublingual administration and dosing for l-epinephrine sublingual tablets. The disclosure provides methods of non-intestinal transmucosal drug delivery of epinephrine, and/or other adrenergic or vasoconstricting pharmaceutical agents (e.g., vasopressin and angiotensin II), that reduce swallowing of drug or saliva-laden drug, which would otherwise reduce bioavailability and efficacy with inconsistent and suboptimal dosing. The present invention may provide oral transmucosal delivery that helps ensure blood perfusion is restored in an emergency situation. Compared to existing methods and compositions, the presently disclosed methods and compositions increase the amount of epinephrine or other active pharmaceutical ingredient that is transported to the circulation inside the tongue, mouth, or cheek to reach the body's systemic circulation. The present disclosure provides methods of elevating plasma epinephrine levels and/or mimicking epinephrine plasma pharmacokinetic parameters/plasma epinephrine levels of an at least one injection of an injectable liquid l-epinephrine formulation, temporarily and/or for an extended period of time, in a human with an at least one dosage of a sublingual l-epinephrine formulation. The presently disclosed methods and formulations provide an improvement over exiting methods and formulations. The presently disclosed methods and formulations may meet an unfilled medical need in a safe, reliable, and comfortable, such as pain-free, manner.

In one embodiment, the present disclosure provides methods of l-epinephrine sublingual administration and dosing for l-epinephrine sublingual tablets that have been proven to be effective in humans in the first-of-its-kind clinical study. This is believed to be the first ever clinical trial conducted of l-epinephrine sublingual tablets; conducted under Good Clinical Practices (GCPs), Good Laboratory Practices (GLPs), and Good Manufacturing Practices (GMPs). Sublingual epinephrine tablets have not been studied in humans before, so there was no idea what to expect. Only non-human preclinical/animal studies were performed previously.

Animal studies of sublingual epinephrine tablets (rabbit studies, Rawas-Qalaji et al, J Allergy Clin Immunol 2006, 117:398-403, as referenced in U.S. Pat. No. 9,877,921 and US 2012/0322884) do not relate to real-life dosing in humans, not only because the sublingual tablets are artificially and mechanically held in position in these animals with laboratory equipment, but because even gentle handling or restraining of small animals greatly increases their plasma levels of catecholamines, and especially increases epinephrine plasma levels (Bühler et al, J Physiol 1978 March, 276: 311-320). Therefore, preclinical animal studies of sublingual epinephrine in the literature cannot and should not be relied upon for human comparison because any non-endogenous dosing can be greatly offset by large spikes in endogenous epinephrine levels released by the adrenal gland in these animals as part of their intrinsic adrenaline ‘fight-or-flight’ response. Other pharmacokinetic parameters would also differ between humans and small animals (e.g., rabbits) when comparing intramuscular injections, and intramuscular injections to sublingual administration, because of the vast size, muscular, and blood volume differences between humans and small animals. A human's mouth and sublingual absorptive area is much greater than that of a rabbit.

Previously, it was believed that the only way to ensure that sublingual epinephrine would be absorbed, rather than ingested, would be to use an adhesive, saliva-impermeable barrier surrounding the sublingual epinephrine formulation on all other sides except the side where the active ingredient portion of the formulation was contacting the mucosal surface in the mouth. This would prevent epinephrine active ingredient from the dissolved tablet from being washed away and ingested.

The present disclosure provides methods of non-intestinal transmucosal drug delivery of sublingual l-epinephrine formulations that reduce the need for such saliva impermeable barriers surrounding the sublingual epinephrine formulation. In an embodiment of these methods, a sublingual l-epinephrine tablet (e.g., a 35 mg to 50 mg l-epinephrine sublingual tablet) is placed under a patient's tongue, such as immediately after removal of the tablet from a dispensing container. The patient is instructed to hold his/her tongue down over the tablet, and to keep his/her tongue and mouth still and not to swallow saliva for at least 5 minutes. The sublingual l-epinephrine tablet may disintegrate within the first 1 minute under the tongue. After 5 minutes, the patient is instructed to swallow once only the excessive saliva above the tongue, while continuing to hold the tongue down over the disintegrated tablet to protect it from mixing with saliva above the tongue and being swallowed. The disintegrated tablet is held under the tongue without swallowing any saliva for an additional 5 minutes. At approximately 10 minutes after the tablet had been placed in the mouth under the tongue, the patient is instructed to swallow all of his/her saliva and any remaining medication under the tongue, and/or the patient can rinse his/her mouth out with a liquid (e.g., water) and spit it out. A second tablet, a third tablet, and so on, can be consecutively administered in this manner. Accordingly, consecutive doses may be spaced apart by at least 10 minutes. For example, consecutive doses may be administered about every 20 minutes. For example, consecutive doses may be administered about every 30 minutes. This disclosure, therefore, includes a method of maintaining a constant/near constant elevated plasma epinephrine level above 40 pg/mL for at least one half hour by the consecutive administration of at least two dosages of an at least one sublingual l-epinephrine formulation.

In other embodiments, consecutive doses are spaced apart by up to 60 minutes or more.

When more rapid epinephrine loading would be helpful, semi-consecutive doses may overlap and be spaced apart by 5 minute increments. The first tablet may be held under the tongue for the full 10 minutes as described above, while the second tablet may be held under the tongue for only 5 minutes (minutes 5 through 10 starting from the first dosage; placed under the tongue after the above-tongue saliva is swallowed), before swallowing everything and/or rinsing the mouth out at 10 minutes from the first dose (5 minutes after the second dose is placed). This scenario may be repeated.

Alternatively, the dosing can be adjusted so that each tablet is held under the tongue for about 5 minutes without any saliva surrounding the tongue being swallowed before then. Then at about 5 minutes, everything can be swallowed and/or the mouth rinsed out, before administering the next tablet; repeated about every 5 minutes.

In some circumstances, more than one tablet may be co-administered simultaneously and held under the tongue. This co-administration may be practiced when someone is losing consciousness and it could be helpful to self-dose, such as if no one else is around. This method may pose greater cardiovascular risk.

In any method of administrating sublingual l-epinephrine tablets, the patient's vitals may be monitored. Another individual may check the patient's breathing and pulse rate. If no one else is around, and if possible, the patient should check his or her own pulse rate. The term ‘patient’ is used, but the recipient of the sublingual l-epinephrine tablet can be anyone (e.g., soldier, policeman, fireman, first responder, student, outdoorsman/hiker, sailor, truck driver, and so on). The patient or individual receiving the sublingual tablet may have his/her head positioned upright, although the sublingual tablet can be administered in a supine position, especially if self-administered.

The above methods employ the use of the tongue to hold the sublingual l-epinephrine formulation down against the floor of the mouth, while the bottom of the tongue covers and surrounds the sublingual l-epinephrine formulation, to seal it off as much as possible from saliva located in the mouth above the tongue from reaching and mixing with the sublingual l-epinephrine formulation under the tongue, so that most or all of the l-epinephrine is absorbed sublingually rather than being ingested with saliva.

These methods of administration are unique because epinephrine has not been administered as a sublingual tablet to humans before. These methods of administration are also unique and nonobvious because other drugs, when in the form of a sublingual tablet, allow swallowing of medicated saliva as soon as the tablet disintegrates under the tongue (e.g., Intermezzo® (zolpidem tartrate) sublingual tablet for treatment of insomnia, Nitrostat® sublingual nitroglycerin tablets for treatment of angina pectoris, and Buprenorphine sublingual tablets for treatment of opioid dependence). There is no hold time for not swallowing medicated saliva with these other drug products. Other sublingual drug products do not employ or suggest the disclosed methods.

The Applicant tested the disclosed methods in the first-of-its-kind human clinical study of l-epinephrine sublingual tablets. This clinical study has not been published and is not publicly available. This was a randomized, crossover, comparator controlled clinical study that measured the bioavailability and descriptive pharmacokinetic data of different strengths of two different l-epinephrine sublingual tablet formulations (one containing l-epinephrine base active pharmaceutical ingredient and one containing l-epinephrine tartrate salt active pharmaceutical ingredient), while recording changes in subject vital signs including blood pressure and heart rate, changes in electrocardiograms, all while looking for signs of mucosal irritation in the mouth. Safety of the tablets was also evaluated. The comparator was an intramuscular injection of 0.3 mg per 0.3 mL of l-epinephrine injection. The study was conducted under fasted conditions. The descriptive pharmacokinetic data or plasma pharmacokinetic parameters included the following: maximum observed plasma concentration, time to maximum observed plasma concentration, area under the plasma concentration versus time curve from time 0 to the last measurable concentration, area under the plasma concentration versus time curve, extrapolated to infinity, terminal elimination rate constant, terminal elimination half-life, apparent clearance, and apparent volume of distribution. Epinephrine was analyzed both with and without baseline correction, as there is endogenous epinephrine present in the body, as well as non-endogenous epinephrine introduced from the tablets. Comparisons were made between the treatment groups.

Some examples of the qualitative and quantitative composition, including percent weight by weight (% w/w), of each component in the l-epinephrine sublingual tablets that were prepared and underwent stability testing are provided in Table 1 and Table 2 below. Table 1 describes the formulations containing Epinephrine Base active pharmaceutical ingredient, while Table 2 describes formulations containing Epinephrine Bitartrate (Salt) active pharmaceutical ingredient. Shown are l-epinephrine sublingual tablet strengths of 2.5 mg, 5 mg, and 50 mg, respectively. Nearly double the amount of Epinephrine Bitartrate (Salt) active pharmaceutical ingredient is required for epinephrine equivalency to Epinephrine Base active pharmaceutical ingredient, so that each 4.548 mg of Epinephrine Bitartrate, USP is equivalent to 2.5 mg of Epinephrine, USP Base; each 9.096 mg of Epinephrine Bitartrate, USP is equivalent to 5 mg of Epinephrine, USP Base; and each 90.960 mg of Epinephrine Bitartrate, USP is equivalent to 50 mg of Epinephrine, USP Base.

TABLE 1
Sublingual tablet formulations made with Epinephrine, USP (Base form)

| | | Quantity | | Quantity | | Quantity | | |
|---|---|---|---|---|---|---|---|---|
| | | per Unit | | per Unit | | per Unit | | |
| | | for | | for | | for | | |
| | | 2.5 mg | | 5 mg | | 50 mg | | Pharmaceutical |
| No. | Component | (mg) | % w/w | (mg) | % w/w | (mg) | % w/w | Function |
| 1 | Epinephrine, | 2.500 | 1.56 | 5.000 | 3.12 | 50.000 | 31.25 | Active Ingredient |
| | USP | | | | | | | |
| 2 | Mannitol, USP | 91.356 | 57.10 | 88.856 | 55.54 | 43.856 | 27.41 | Diluent |
| | (Mannogem EZ | | | | | | | |
| | Spray Dried) | | | | | | | |
| 3 | Microcrystalline | 56.000 | 35.00 | 56.000 | 35.00 | 56.000 | 35.00 | Diluent |
| | Cellulose, NF | | | | | | | |
| | (Avicel PH-102) | | | | | | | |
| 4 | Crospovidone | 8.000 | 5.00 | 8.000 | 5.00 | 8.000 | 5.00 | Disintegrating |
| | (Type-A), NF | | | | | | | Agent |
| | (Polyplasdone | | | | | | | |
| | XL) | | | | | | | |
| 5 | Sucralose, NF | 0.544 | 0.34 | 0.544 | 0.34 | 0.544 | 0.34 | Sweetening Agent |
| 6 | Magnesium | 1.600 | 1.00 | 1.600 | 1.00 | 1.600 | 1.00 | Lubricant |
| | Stearate, NF | | | | | | | |

| | | | | | | |
|---|---|---|---|---|---|---|
| Total weight | 160.000 | 100.00 | 160.000 | 100.00 | 160.000 | 100.00 |

TABLE 2
Sublingual tablet formulations made with Epinephrine Bitartrate, USP (Salt form)

| | | Quantity | | Quantity | | Quantity | | |
|---|---|---|---|---|---|---|---|---|
| | | per Unit | | per Unit | | per Unit | | |
| | | for | | for | | for | | |
| | | 2.5 mg | | 5 mg | | 50 mg | | Pharmaceutical |
| No. | Component | (mg) | % w/w | (mg) | % w/w | (mg) | % w/w | Function |
| 1 | Epinephrine | 4.548 | 2.84 | 9.096 | 5.69 | 90.960 | 56.85 | Active Ingredient |
| | Bitartrate, USP | | | | | | | |
| 2 | Mannitol, USP | 89.308 | 55.82 | 84.760 | 52.98 | 2.896 | 1.81 | Diluent |
| | (Mannogem EZ | | | | | | | |
| | Spray Dried) | | | | | | | |
| 3 | Microcrystalline | 56.000 | 35.00 | 56.000 | 35.00 | 56.000 | 35.00 | Diluent |
| | Cellulose, NF | | | | | | | |
| | (Avicel PH-102) | | | | | | | |
| 4 | Crospovidone | 8.000 | 5.00 | 8.000 | 5.00 | 8.000 | 5.00 | Disintegrating |
| | (Type-A), NF | | | | | | | Agent |
| | (Polyplasdone | | | | | | | |
| | XL) | | | | | | | |
| 5 | Sucralose, NF | 0.544 | 0.34 | 0.544 | 0.34 | 0.544 | 0.34 | Sweetening Agent |
| 6 | Magnesium | 1.600 | 1.00 | 1.600 | 1.00 | 1.600 | 1.00 | Lubricant |
| | Stearate, NF | | | | | | | |

| | | | | | | |
|---|---|---|---|---|---|---|
| Total weight | 160.000 | 100.00 | 160.000 | 100.0 | 160.000 | 100.00 |

The manufacturing of the above tablets included the steps of dispensing, sifting, blending, and compression/tableting. Briefly, these tablets were prepared by dispensing and sifting active ingredient and some quantities of inactive ingredients with a sieve to make a first premix blend. Other quantities of inactive ingredient were dispensed and sifted with a sieve to make a second premix blend. Magnesium Stearate and mannitol were also sifted with a sieve. Several rounds of blending with a blender for up to 10 minutes were applied as the first sifted premix blend was added, followed by the second sifted premix blend, as well as additional sifted mannitol and sifted magnesium. The final blend was compressed into tablets with a multi-station tablet press. Tablets were packed into bottles with desiccant and labeled. Initially, 100 count tablet bottles were prepared and placed in stability chambers, including with accelerated conditions at 40 degrees Celsius. The tablets were tested by analytical laboratory methods during development, at release, and during the stability testing program. These tablets were found to be very stable with good l-epinephrine purity profiles and low impurities. These tablets were named, and bottles labeled as, ADRENATAB.

The l-epinephrine active pharmaceutical ingredients used to make these tablets (ADRENATAB tablets), i.e., Epinephrine Base active pharmaceutical ingredient and Epinephrine Bitartrate Salt active pharmaceutical ingredient, come supplied from their manufacturer as a powder with particles in the micrometer range; i.e., are mostly microparticles of epinephrine. Human bioavailability was found to be good with these tablets and the disclosed methods, and the tablets and methods are improvements over known tablets and methods. By way of example, the patent references of Rawas-Qalaji et al U.S. Pat. No. 9,877,921 and US 2012/0322884 describe “Epinephrine nanoparticles, methods of fabrication thereof, and methods for use thereof for treatment of conditions responsive to epinephrine.” Rawas-Qalaji et al's methods of making epinephrine nanoparticles involve combining epinephrine with solvents, such as isopropyl alcohol, to form a mixture, which is then subjected to high pressure and temperature in a reaction chamber. Any marginal increase in absorption, if any, by making these nanoparticles would be countered by the creation of additional impurities, and especially the impurity dextrorotary-(d)-epinephrine that forms as levorotary-(l)-epinephrine racemizes in solvent and with temperature and pressure. Rawas-Qalaji et al does not address these issues of epinephrine racemization and does not check chirality. Moreover, Rawas-Qalaji et al uses excipients, such as chitosan, which appear not to be pharmaceutically acceptable. Chitosan is not found in the FDA's inactive ingredient database for approved drug products. Chitosan is derived from shellfish origin to which individuals may be highly allergic. Creating an epinephrine tablet with an ingredient that may cause severe allergic reaction, e.g., chitosan, is counterproductive and counterintuitive when a purpose of the tablet is to treat severe allergic reactions in allergy-susceptible individuals. e disclosed tablets and methods are an improvement over those of Rawas-Qalaji et al.

Pharmacokinetic results of the presently disclosed first human clinical trial demonstrated, based on the point estimates observed for baseline corrected epinephrine, that the rate of absorption (Cmax) is lower for tablets having the Tartrate Salt form of epinephrine when compared to those having the Base form of epinephrine, while the extent of absorption (AUC 0-t) was similar. As mentioned, the l-epinephrine sublingual tablets used in this human clinical study were named, and bottles labeled as, ADRENATAB™. ADRENATAB l-epinephrine sublingual tablets are identified in the Figures as either ADRENATAB Base (tablets made with l-epinephrine base active ingredient) or ADRENATAB Tartrate Salt (tablets made with l-epinephrine bitartrate [salt]). In the study, blood samples were collected for analysis at 1 hour before and at a half hour before dosing; at time zero; and at 5 minutes, 10 minutes, 15 minutes, 20 minutes, 30 minutes, 40 minutes, 1 hour, 1.5 hours, 2 hours, 3 hours, and 6 hours after tablet dosing. Plasma epinephrine concentrations were measured by a bioanalytical facility using validated methods.

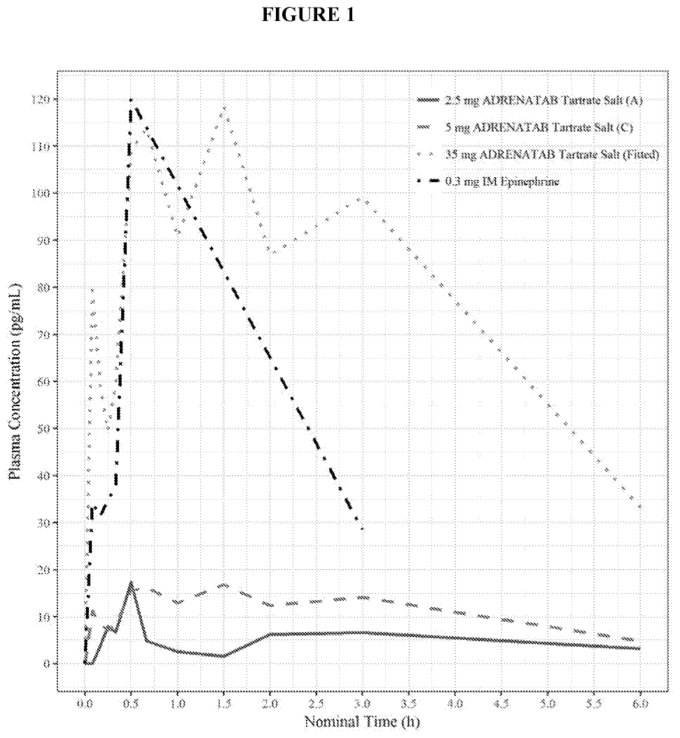

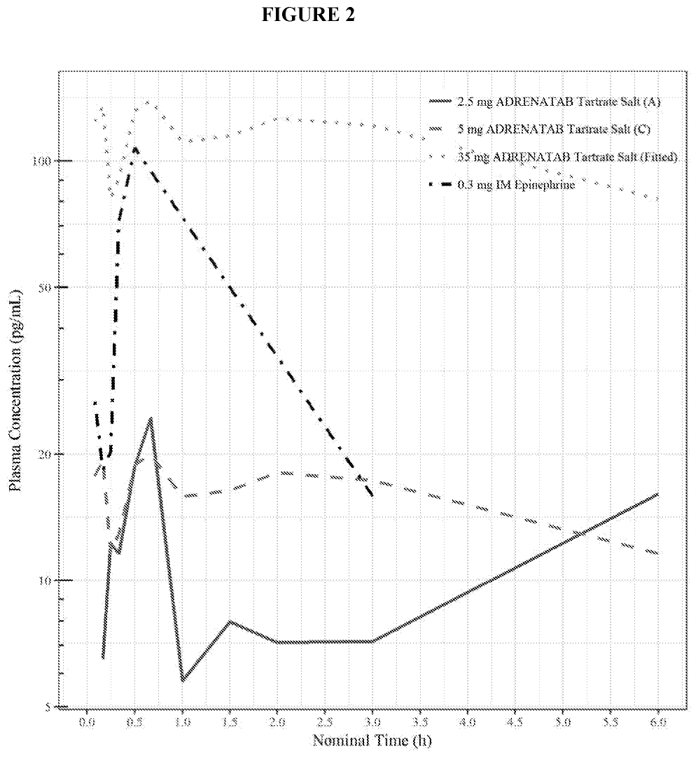

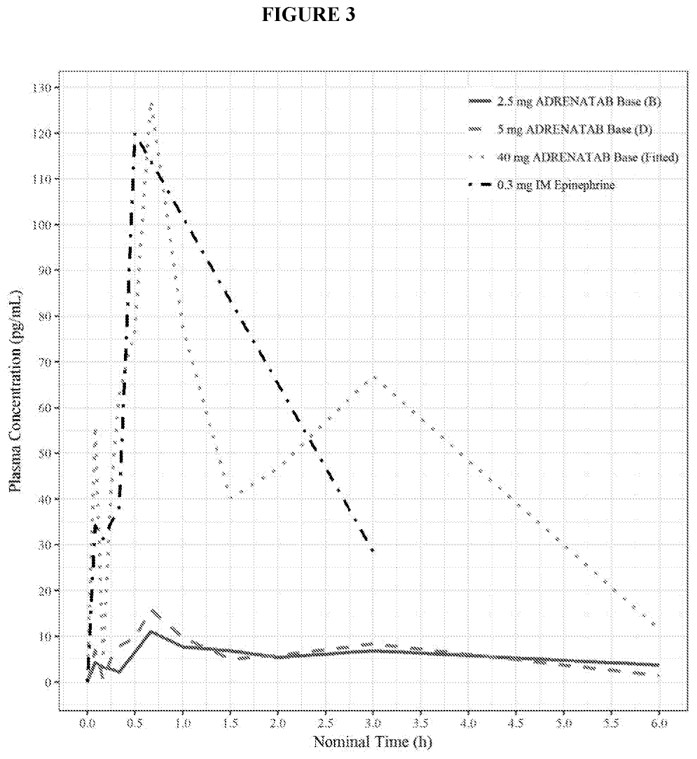

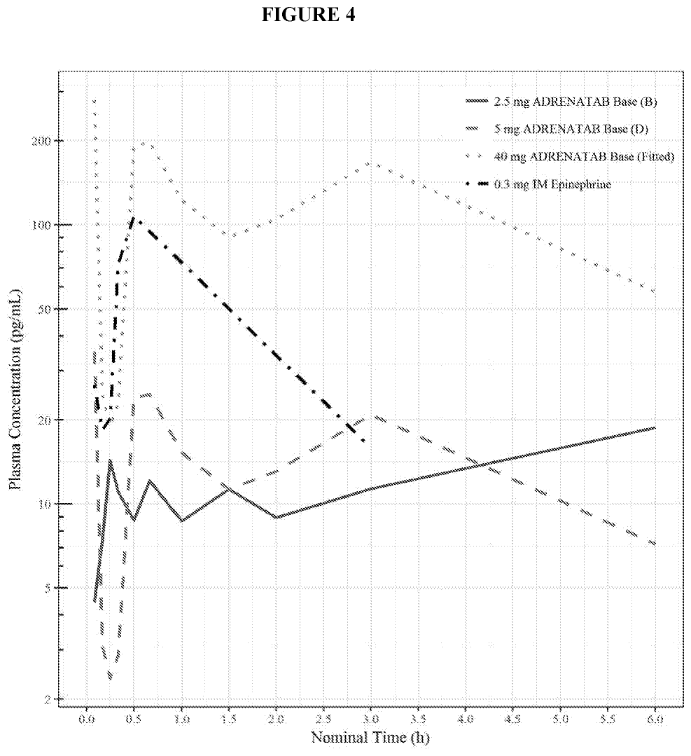

FIGS. 1-4 show mean baseline corrected epinephrine plasma concentrations (pg/mL) plotted against nominal time (hours) for subjects dosed with ADRENATAB l-epinephrine sublingual tablets compared to subjects dosed with a 0.3 mg intramuscular epinephrine injection into the lateral part of the thigh. Two of the lower ADRENATAB tablet strengths of 2.5 mg and 5 mg are shown in these figures for reference to demonstrate that absorption took place even at these low dosages. Also shown is a calculated ADRENATAB tablet dose that best fits the mean baseline corrected epinephrine plasma concentrations following a 0.3 mg intramuscular (IM) epinephrine injection. For tablets made with Epinephrine Bitartrate, this fitted dose was calculated to be 35 mg, while for tablets made with Epinephrine Base, this fitted dose was calculated to be 40 mg. FIG. 1 and FIG. 2 show results of subjects dosed with the Epinephrine Bitartrate (Tartrate Salt) formulation of tablets, while FIG. 3 and FIG. 4 show results of subjects dosed with the Epinephrine Base formulation of tablets. FIG. 1 and FIG. 3 are shown in linear scale, while FIG. 2 and FIG. 4 are shown in logarithmic (Log) scale.

The disclosed methods of mimicking epinephrine plasma pharmacokinetic parameters/plasma epinephrine levels of an at least one injection of an at least one injectable liquid l-epinephrine formulation in humans with an at least one dosage of an at least one sublingual l-epinephrine formulation have been tested with the above clinical study conducted in humans. Pharmacokinetic data from this human clinical study demonstrates that elevated plasma epinephrine levels, including non-endogenous epinephrine, can be sustained for an extended period of time by the administration of l-epinephrine sublingual tablet(s).

The disclosed methods may help protect and prevent drug from mixing with saliva and being swallowed, thereby minimizing or preventing both decreased bioavailability and loss of efficacy, and ensuring restoration of proper blood perfusion with the activity of the drug. The drug may be l-epinephrine and/or other adrenergic or vasoconstricting pharmaceutical agents (e.g., norepinephrine, vasopressin, and angiotensin II). Some other future and/or novel sublingual drugs may be administered using the disclosed methods, such as sublingual insulin or an insulin analogue, insulin derivative, proinsulin, or drug that mimics insulin or insulin signaling, or a combination thereof.

In one embodiment, a method of mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation is provided. The at least one injectable liquid l-epinephrine formulation may be at a concentration of 0.1 mg per mL to 1 mg per mL prior to any diluting. The at least one injectable liquid l-epinephrine formulation may contain 0.1 mg to 1 mg of l-epinephrine and/or the at least one injectable liquid l-epinephrine formulation may contain 0.1 mL to 10 mL of l-epinephrine prior to any diluting. The at least one injection of an at least one injectable liquid l-epinephrine formulation may be selected from endotracheal injection, intracardiac injection, intramuscular injection, subcutaneous injection, subcutaneous infusion, intravenous injection, and intravenous infusion.

Such injectable liquid l-epinephrine formulations are administered to treat individuals and/or patients who are experiencing any one or more of the following: hypotension, such as may occur from drug or allergen hypersensitivity reactions; anaphylaxis; anaphylactic shock; sepsis; septic shock; respiratory distress, such as may occur by bronchospasm, asthma/asthma attack, or respiratory infection; inadequate heart rhythm, such as may occur in cardiac arrest, arrhythmia, syncope/heart block, and those needing resuscitation; and mucosal congestion, such as may occur from allergy or sinus infection. In some instances, such injectable liquid l-epinephrine formulations can also be administered to prophylactically treat individuals and/or patients who are experiencing any of the above indications. The disclosed methods can achieve the above treatments and provide similar physiologic (pharmacodynamic) effects using sublingual l-epinephrine instead of an injection.

In most embodiments and methods, the at least one sublingual l-epinephrine formulation contains at least 10 mg of l-epinephrine and no more than 200 mg of l-epinephrine. In many embodiments and methods, the at least one sublingual l-epinephrine formulation contains at least 25 mg of l-epinephrine and no more than 70 mg of l-epinephrine.

In some embodiments, the at least one sublingual l-epinephrine formulation is in sublingual tablet form, which may be an orally disintegrating sublingual tablet form.

In some embodiments, the at least one sublingual l-epinephrine formulation is in sublingual tablet form and contains at least one, or at least two, pharmaceutically acceptable excipient ingredients.

In some embodiments, the at least one sublingual l-epinephrine formulation is in sublingual tablet form and contains at least one diluent excipient ingredient, at least one disintegrating agent excipient ingredient, at least one lubricant excipient ingredient, and optionally, at least one sweetening agent/taste masking agent excipient ingredient and/or colorant.

In some embodiments, the at least one sublingual l-epinephrine formulation contains l-epinephrine active pharmaceutical ingredient that is not exposed to solvent and not exposed to drying processes during production/manufacturing of said sublingual l-epinephrine formulation. In other words, the l-epinephrine active pharmaceutical ingredient is not wetted and not exposed to solvents that would otherwise at least partially dissolve the l-epinephrine active pharmaceutical ingredient, which would require heat or drying to form a non-liquid formulation, and cause oxidation and racemization, i.e., degradation. Optionally, the l-epinephrine active pharmaceutical ingredient does not contain, or is not essentially comprised of, nanoparticles.

In some embodiments, the at least one sublingual l-epinephrine formulation contains less than 6% d-epinephrine impurity at release and contains less than 12% d-epinephrine impurity over a shelf-life of at least 12 months, and in most instances, longer.

In some embodiments, the at least one sublingual l-epinephrine formulation has a disintegration time of no more than 2 minutes.

In some embodiments, the at least one sublingual l-epinephrine formulation has a disintegration time of no more than 1 minute, and may be well under 1 minute.

In one embodiment, a method of elevating and mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation is provided. This method further includes a first step of removing/dispensing the at least one dosage of the at least one sublingual l-epinephrine formulation from a dispensing container and placing the at least one dosage of the at least one sublingual l-epinephrine formulation under a human's tongue; this method further includes a second step of the human holding his/her tongue down over the at least one dosage of the at least one sublingual l-epinephrine formulation, while keeping the tongue and mouth still to protect the at least one dosage of the at least one sublingual l-epinephrine formulation from mixing with saliva above the tongue, and not swallowing any saliva for at least 5 minutes; this method further includes an optional third step of placing another at least one dosage of an at least one sublingual l-epinephrine formulation under the human's tongue.

In another embodiment, a method of elevating and mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation is provided. This method further includes a first step of removing/dispensing the at least one dosage of the at least one sublingual l-epinephrine formulation from a dispensing container and placing the at least one dosage of the at least one sublingual l-epinephrine formulation under a human's tongue. This method further includes a second step of the human holding his/her tongue down over the at least one dosage of the at least one sublingual l-epinephrine formulation, while keeping the tongue and mouth still to protect the at least one dosage of the at least one sublingual l-epinephrine formulation from mixing with saliva above the tongue, and not swallowing any saliva for at least 5 minutes. This method further includes a third step of swallowing once (one time) any excessive saliva above the tongue, while continuing to hold the tongue down over the at least one dosage of the at least one sublingual l-epinephrine formulation; holding of the tongue down over the at least one dosage of the at least one sublingual l-epinephrine formulation continues for up to an additional 5 minutes. This method further includes a fourth step of mouth-clearing by swallowing all saliva and/or mouth contents, and/or rinsing the mouth out with a liquid and spitting. This method is optionally repeated as is needed or desired. This method was applied to dosing in the human clinical study mentioned above.

Some embodiments include a method of elevating and/or mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation. At least two dosages of the at least one sublingual l-epinephrine formulation may be administered at least 5 minutes apart.

The disclosure includes a method of maintaining a constant/near constant elevated plasma epinephrine level above 40 pg/mL in humans for at least one half hour by the consecutive administration of at least two dosages of an at least one sublingual l-epinephrine formulation.

The disclosure includes a method of maintaining a constant/near constant raise in blood pressure by the administration of an at least one dosage of an at least one sublingual l-epinephrine formulation. The disclosure also includes a method of maintaining a constant/near constant raise in blood pressure for at least one half hour by the consecutive administration of at least two dosages of an at least one sublingual l-epinephrine formulation. This method may normalize, or nearly normalize, a hypotensive state to a normotensive blood pressure. This method may increase blood pressure by a few mmHg, which may help reduce the loss of perfusion.

The disclosure includes a method of repeat dosing of an at least one sublingual l-epinephrine formulation to elevate plasma epinephrine levels to maintain at least one of blood pressure, pulse, and breathing in a human patient having at least one of anaphylaxis, anaphylactic shock, sepsis, septic shock, respiratory difficulty, and cardiac difficulty, when an at least one injection of an injectable liquid l-epinephrine formulation is not available/not possible, at least until emergency medical services arrive to treat/transport the human patient. The human patient may be a soldier on a battlefield/remote location. The at least one injection of an injectable liquid l-epinephrine formulation may be administered later if available/possible.

The Applicant's methods may help ensure that most or more than half (more than 50%) of the active pharmaceutical ingredient of l-epinephrine from the pharmaceutical formulation of epinephrine is absorbed, without considerable racemization, and is prevented from being swallowed. The methods involve the tongue holding a formulation against a mucosa surface and blocking out all, nearly all, or most other saliva from surrounding areas/vicinities. Saliva from surrounding areas should not interact to a considerable extent. Alternative methods also couple these methods with a saliva impermeable barrier in the formulation and/or associated with the formulation. In alternative embodiments, the barrier is chosen from physical structures including a non-absorbable removable layer of plastic or polymer material that provides a physical surface to which the pharmaceutical formulation of epinephrine is attached, at least temporarily, the physical structure further protecting/sheathing the pharmaceutical formulation of epinephrine from mixing with saliva outside of a zone/region where the pharmaceutical formulation of epinephrine is contacting and/or secured/adhered to the mucous membrane.

In embodiments, the sublingual l-epinephrine formulation contains no chitin, as chitin is derived from shellfish, which could induce an allergic reaction in susceptible individuals. In embodiments, the sublingual l-epinephrine formulation contains no sulfites (e.g., no metabisulfite), which could induce an allergic reaction in susceptible individuals.

Some embodiments include no tartrate or bitartrate in the sublingual l-epinephrine formulation as epinephrine base may perform better.

In some embodiments, the sublingual l-epinephrine formulation is associated with an at least one bioadhesive.

The disclosure also provides a method of elevating plasma epinephrine levels (above normal baseline levels, or up to 40 pg/mL and/or above 40 pg/mL) in a human with an at least one dosage of a sublingual l-epinephrine formulation. Embodiments include sublingual l-epinephrine orally disintegrating tablets. The disclosure includes methods of elevating plasma epinephrine levels for an extended period of time, such as up to a half hour, or even beyond a half hour, by the administration of an at least one dosage of a sublingual l-epinephrine formulation. The disclosure also provides a method of elevating plasma epinephrine levels (above normal baseline levels, or up to 40 pg/mL and/or above 40 pg/mL) in a human with an at least one dosage of a sublingual l-epinephrine formulation containing at least one additional active pharmaceutical ingredient other than epinephrine, or an at least one dosage of a sublingual l-epinephrine formulation consecutively administered and/or co-administered with an at least one formulation containing an active pharmaceutical ingredient other than epinephrine.

The disclosure also provides a method of elevating plasma epinephrine levels, and/or a method of mimicking epinephrine plasma pharmacokinetic parameters/plasma epinephrine levels of an at least one injection of an injectable liquid l-epinephrine formulation, temporarily and/or for an extended period of time (e.g., a half hour), in a military soldier and/or military pilot with an at least one dosage of a sublingual l-epinephrine formulation. The disclosed methods and at least one dosage of a sublingual l-epinephrine formulation can be used to not only treat a soldier experiencing at least one of hypotension, respiratory difficulty, and/or cardiac difficulty, but also to enhance the physical and/or mental training of soldiers, and also to stimulate soldiers during extreme combat situations.

Soldiers, like other individuals, may be sensitive to food allergens and insect bites, which can trigger hypersensitivity reactions or anaphylaxis in susceptible individuals. The disclosure reduces or negates the need for soldiers to carry around bulky and heat-sensitive liquid formulations of epinephrine, whether as an epinephrine autoinjector or epinephrine syringes, vials, or ampoules. Instead, a tiny container of one or more sublingual l-epinephrine tablets may be carried.

Compared to most individuals, soldiers are at greater risk of being exposed to many other chemical agents that may trigger anaphylaxis, whether it be from smoke inhalation of the burning of noxious materials or even from chemical warfare agents. The ATROPEN® is a prefilled auto-injector of Atropine Injection, an anticholinergic agent used to treat poisoning by susceptible organophosphorous nerve agents having cholinesterase activity as well as organophosphorous or carbamate insecticides. Atropine injection is believed to be part of an antidote kit prepared for the military. Likewise, sublingual l-epinephrine formulations, including tablets such as ADRENATAB, could be included in an antidote kit for soldiers. The disclosed methods include the sequential dosing and/or co-administration of an at least one sublingual l-epinephrine formulation and an at least one atropine and/or hyoscyamine formulation, or other anticholinergic or antidote agent(s). The atropine (or its levorotary isomer hyoscyamine) may be administered as a liquid injection and/or as a solid oral formulation, and may be a sublingual formulation. In another embodiment, a combination sublingual formulation is administered containing both l-epinephrine active ingredient and atropine (and/or hyoscyamine) active ingredient as an antidote to nerve poisoning agent(s), or containing other anticholinergic or antidote agent(s). These examples are not meant to be limiting.

The disclosed sublingual l-epinephrine formulation or dosage form and methods can also be used to enhance the physical and/or mental training of combat soldiers and even combat vehicle drivers or fighter pilots by boosting blood perfusion throughout the brain and body with higher cardiac output, as well as increasing brain metabolism and oxygen consumption by the brain. The disclosure may also simulate an ‘adrenaline rush’ during training that a soldier or pilot would otherwise experience out in the field. This may beforehand demonstrate the soldier's or pilot's ability to operate under the mental pressure and physical stress of combat or tactical missions.

Additionally, the disclosed sublingual l-epinephrine formulation or dosage form and methods can be used to stimulate soldiers and/or pilots during extreme combat situations. Such stimulation is believed to reduce reaction time and reduce fear, and even temporarily reduce pain, such as from a wound (e.g., bullet wound). This may be useful if a soldier charges several combatants in a high-risk situation or is in an intense dog fight with multiple combatants. With these methods, a soldier may impose maximum damage on the enemy. These methods may provide at least some chance of survival in what would otherwise be a ‘suicide mission’. The disclosed methods may also include the sequential dosing and/or co-administration of an at least one sublingual l-epinephrine formulation and the administration of an at least one stimulant and/or analgesic. The at least one stimulant and/or analgesic may be administered as liquid injection(s), topical/transdermal application(s), and/or solid oral formulation(s), or as sublingual formulation(s). In another embodiment, a combination sublingual formulation is administered containing both l-epinephrine active ingredient and stimulant active ingredient (e.g., an amphetamine, e.g., a xanthine [caffeine or cocaine analogue]) and/or analgesic active ingredient (e.g., a member of the opioid class, an opioid, such as morphine or fentanyl, or another opioid analogue, derivative, or a combination thereof) to enhance the ability of the soldier and/or pilot during a combat situation. These examples are not meant to be limiting and other members of stimulant and/or analgesic classes can be substituted or additionally included.

The disclosed sublingual l-epinephrine formulation or dosage form and methods can also be used to prevent, delay, or reduce the time that a combat pilot, or even astronaut, may lose consciousness (i.e., blackout) from high acceleration or gravitational force (e.g., more than a several-fold g-force or a maximum g-force).

Still further, the disclosed sublingual l-epinephrine formulation or dosage form and methods can help revive a soldier after experiencing physical trauma or sedation.

The disclosure also provides a method of elevating plasma epinephrine levels, and/or a method of mimicking epinephrine plasma pharmacokinetic parameters/plasma epinephrine levels of an at least one injection of an injectable liquid l-epinephrine formulation, temporarily and/or for an extended period of time (e.g., a half hour), in an athlete with an at least one dosage of a sublingual l-epinephrine formulation. These methods can help revive an athlete experiencing exercise induced hypotension, bronchospasm, asthmatic episode or cardiovascular collapse. Further, the disclosed sublingual l-epinephrine formulation or dosage form and methods can also be used to enhance the physical and/or mental training of athletes by boosting blood perfusion throughout the brain and body with higher cardiac output, as well as increasing brain metabolism and oxygen consumption by the brain. The disclosure may also simulate an ‘adrenaline rush’ during athletic training that an athlete would otherwise experience out in the field during a game, race, or other athletic event or competition. Where permitted, when not considered ‘doping’, the disclosed sublingual l-epinephrine formulation or dosage form and methods can also be used to stimulate (professional) athletes or Olympians during an event to enhance performance, reduce reaction time and reduce fear, and even temporarily reduce pain. Epinephrine may increase blood flow to muscles and increase blood glucose levels, along with cardiac output, which may enhance athletic performance.

Further, the disclosure also provides a method of elevating plasma epinephrine levels, and/or a method of mimicking epinephrine plasma pharmacokinetic parameters/plasma epinephrine levels of an at least one injection of an injectable liquid l-epinephrine formulation, temporarily and/or for an extended period of time (e.g., a half hour), for neurocognitive enhancement, especially if combined with a ‘smart drug,’ ‘smart supplement’, or other cognitive enhancer (a ‘nootropic’ agent). Such nootropic agents may include supplements such as ginseng and Ginkgo biloba, and drugs such as racetams, e.g., piracetam and its derivatives. Nootropic agents can also include amphetamines (like Adderall® and Ritalin®), methylphenidate, modafinil, nicotine and their analogues, derivatives, and combinations thereof. These examples are not meant to be limiting. At least temporary neuroenhancement with the disclosed methods may help in thinking of better strategies and solutions to problems in science, medicine, business, warfare, and help ‘think tanks’ and innovation as well. These methods can enhance blood flow and oxygen utilization by the brain and stimulate synaptic activity.

The above examples are meant to show that there can be additional properties and/or synergies with the disclosed methods and/or of combining the disclosed methods with one or more additional active ingredients.

The disclosed methods even allow for a ‘cardiac stress test in a bottle’, as a method of simulating and/or enhancing what is known as a cardiac diagnostic test/cardiopulmonary exercise test that usually requires response to an external stress (e.g., treadmill exercise) in a controlled environment. The disclosed methods mimic the increase in plasma epinephrine levels that occurs with exercise, and/or mimics changes to a subject's vital signs that occur with exercise, by administering an at least one dosage of a sublingual l-epinephrine formulation according to the disclosure.

In embodiments, the pharmaceutical formulation of l-epinephrine is adapted to be immediately released for rapid transmucosal delivery. Other embodiments provide an extended release/sustained release sublingual l-epinephrine formulation or dosage form. A slow release or controlled release embodiment can contain up to 1,000 mg or more of epinephrine slowly released, such as to mimic intravenous infusion of epinephrine, such as for treating hypotension associated with septic shock. There can be other disease states, such as those associated with adrenal insufficiency, that may require slow, titrated epinephrine administration.

Other embodiments can include a dosage form that is a chewing gum, gummy candy, or hard candy/lozenge, or a sublingual breath spray. In still other embodiments, a patch (e.g., buccal patch) may be utilized, such as an epinephrine buccal patch. The tongue may be able to hold and cover a formulation to block saliva in the mouth from mixing with the formulation in other positions in the oral cavity. The at least one mucous membrane inside the patient's mouth is chosen from at least one mucosal site, including, but not limited to, sublingual, sublabial, buccal, palate, and gingiva sites in the patient's mouth.

In some embodiments, the sublingual l-epinephrine formulation is optionally surrounded by a saliva-impermeable physical barrier or excipient formulation on all sides except for the one side that contacts the sublingual mucosa where absorption takes place. This saliva-impermeable physical barrier further protects the l-epinephrine active ingredient from interacting with saliva beyond the site of attachment. This physical structure or barrier, in some embodiments, can also help adhere the pharmaceutical formulation to the oral mucosa site and help keep it dry from saliva. In some of these embodiments, this protective layer or physical structure, such as a film, membrane, or gel, dissolves to release the pharmaceutical active ingredient for delivery, and/or dissolves after the active pharmaceutical ingredient has been delivered transmucosally. The timing of such dissolving of this protective layer or physical structure can be modified to achieve the desired dosing effect, and as such, the size, thickness, and composition of the protective layer or physical structure is chosen as such. In some embodiments, this protective layer or physical structure is bioabsorbable or degradable. In yet other embodiments, this protective layer or physical structure is non-absorbable, such as a plastic polymer, and instead may be physically removed from the oral cavity following transmucosal drug delivery, or may be swallowed and excreted by the patient following transmucosal drug delivery inside the oral cavity. When a non-absorbable protective layer or physical structure is adhered to or around the target mucosa, a warm liquid, such as water or alcohol may be required for its removal, along with rubbing the protective layer or physical structure with a finger, brush, utensil, washcloth, or another object to loosen the adhesive. In other embodiments, the adhesion can be negated by a change in pH, exposure to air, or other biologically safe solvents. In some embodiments, this protective layer or physical structure blocks at least some of the flavor of bad/bitter tasting pharmaceutical ingredients, and as such, can also minimize or prevent additional saliva secretion from taking place when an otherwise bad/bitter taste is experienced.

These methods provide a more consistent and reproducible dosage, with greater bioavailability and less metabolic/enzymatic degradation over prior methods of transmucosal delivery in the oral cavity. These methods are further associated with at least one of greater systemic bioavailability, lower dilution, lower side effects, or a combination thereof.

The pharmaceutical formulation of the disclosure includes at least one dosage form chosen from the class of dosage forms including, but not limited to, a liquid, liquid droplets, a gel, a paste, a film, a biofilm, a tape, a wafer, a membrane, a sheath, a disc, beads, a tablet, a caplet, lozenges, semi-solids, glues, gums, or a combination thereof. Capsules are also possible, such as capsules containing l-epinephrine active ingredient powder. The pharmaceutical formulation further includes at least one pharmaceutically acceptable excipient selected from pharmaceutical excipients including, but not limited to, carriers, solvents, solubilizing excipients, taste-masking excipients, sweeteners, lipids, liposomes, adherents, bioadhesives, glycoproteins, proteins, carbohydrates, starches, waxes, polymers, charged/ionic excipients, absorption enhancing excipients, non-charged/neutral excipients, nanoexcipients/nanoparticles, microencapsulating excipients, chelating excipients, excipients that dissolve over time, biocompatible excipients, non-absorbable excipients, and bioabsorbable excipients. The at least one pharmaceutically acceptable excipient is further chosen for the desired speed of transmucosal delivery, whether desired for immediate release and delivery, or slow release and sustained transmucosal delivery. The at least one pharmaceutically acceptable excipient may be chosen to aid in the passive diffusion and/or active transport of the at least one pharmaceutical active ingredient.

This disclosure also includes at least two dosages of a pharmaceutical formulation. These two dosages may be two different dosage amounts. These at least two different dosages, in some embodiments, may have different releasing characteristics, for example, with layers or emulsions of excipients chosen for timed release or differential release. These at least two different dosages, in other embodiments, may have different size/area profiles and/or different concentrations per square unit of area in contact with a mucosal surface. Furthermore, each different dosage or dosage amount can be specific to a certain mucosal site in the oral cavity so that different dosages can be administered to different mucosae in the oral cavity. This method can allow the sites in the mouth to be rotated, so that the same mucosa site is not being dosed over and over again, so as to avoid local complications, such as localized toxicity or reduced transport at that site during a treatment regimen. Because transport efficiency and the vasculature may differ between different mucosae in the oral cavity, e.g., the palate versus buccal sites, dosages can be calibrated for these various mucosae. This method of dosage and mucosa site rotation can maintain efficient drug transport over a treatment regimen, whether formulated for active transport or passive diffusion, and whether formulated for immediate release or sustained transmucosal delivery.

In some embodiments, a collective dose is formed of smaller dosage aliquots comprised of one or more dosages from one or more different applicators. Combinations of different dosage amounts and different applicators provide different options to patients to ensure that certain mucosa are not being over-treated with drug so as to reduce local side effects and to provide optimum bioavailability. These different applicator combinations can be rotated to provide the same collective (total) dose, without treating the same mucosa sites consecutively.

Another embodiment includes a method of non-intestinal transmucosal delivery of a pharmaceutical formulation of l-epinephrine, including an active pharmaceutical ingredient of l-epinephrine and at least one pharmaceutically acceptable excipient, the method avoiding at least most first-pass metabolism by the liver by limiting or preventing saliva from mixing with this active pharmaceutical ingredient of l-epinephrine and being ingested. The method includes administration of this pharmaceutical formulation of l-epinephrine inside a patient's mouth for transmucosal delivery across an at least one mucous membrane inside the patient's mouth. This method further includes adhering the pharmaceutical formulation of l-epinephrine to the at least one mucous membrane with a bioadhesive for transmucosal delivery. In some embodiments, the bioadhesive is a mucoadhesive. This method still further includes sheathing the pharmaceutical formulation of l-epinephrine with a barrier to protect from mixing with saliva outside of a zone/region where the pharmaceutical formulation of l-epinephrine is adhered to the at least one mucous membrane, so that more than half (more than 50%) of the active pharmaceutical ingredient of l-epinephrine from the pharmaceutical formulation of l-epinephrine is prevented from being swallowed. The barrier may include a hydrophobic layer, hydrophobic surface, physical barrier, or combination thereof at least partially surrounding the pharmaceutical formulation of l-epinephrine. This method is further associated with at least one of greater systemic bioavailability, lower dilution, lower side effects, or a combination thereof.

Further embodiments of the disclosure also include placing and adhering the pharmaceutical formulation to mucosa inside the oral cavity. The disclosure also includes an applicator to aid in the placement of the pharmaceutical formulation inside the mouth and may aid in adhering the formulation to the target mucosa site. In some embodiments, the applicator is used to administer a single dosage of said pharmaceutical formulation. In other embodiments, the applicator is used to administer multiple dosages of one or more pharmaceutical formulations. In some of these embodiments, the applicator houses and dispenses multiple dosages of one or more pharmaceutical formulations of the disclosure with the bioadhesive.

In some embodiments, l-epinephrine is accompanied with or replaced by an at least one other member of the class of adrenergic or vasoconstricting pharmaceutical agents. Examples of other adrenergic or vasoconstricting pharmaceutical agents of this class include norepinephrine, vasopressin, and angiotensin II. In other embodiments, l-epinephrine is accompanied by an at least one other pharmaceutical agent in the same pharmaceutical formulation or in a separate pharmaceutical formulation. In some other embodiments, l-epinephrine is accompanied by an at least one other pharmaceutical agent selected from the class of antihistamines, e.g., diphenhydramine, either in the same pharmaceutical formulation or in a separate pharmaceutical formulation.

Embodiments of this disclosure can include various numbers of excipient ingredients and/or percent weight/weight of these excipient ingredients. Excipient ingredients are selected from the classes of excipients including, but not limited to, antiadherents, binders, coatings, nanoparticles, chelators, buffering agents, acid reacting excipients, alkaline reacting excipients, disintegrants, fillers, diluents, colors/colorants, lubricants, glidants, preservatives, sorbents, absorption enhancers, flavors, sweeteners, carriers, solvents, surfactants, bioadhesives, mucoadhesives, and any mixtures and combinations thereof; and can include lipids, liposomes, glycoproteins, proteins, carbohydrates, saccharides, starches, waxes, and polymers.

An at least one bioadhesive excipient is an element and/or an additional element to some methods, which includes adhering the pharmaceutical formulation of at least one active pharmaceutical ingredient, which in embodiments is l-epinephrine, to at least one oral mucous membrane with the at least one bioadhesive for transmucosal delivery of said at least one active pharmaceutical ingredient. The active pharmaceutical ingredient (e.g., l-epinephrine) portion of the drug product may be secured or adhered directly or indirectly to the oral mucosal membrane. Generally, the active pharmaceutical ingredient (e.g., l-epinephrine) portion of the drug product is secured in contacting abutment to the oral mucosal membrane by surrounding bioadhesive of the drug product or physical structure—an indirect adhesion of the active pharmaceutical ingredient. In some instances, however, some bioadhesive may exist in the active pharmaceutical ingredient-containing formulation, allowing direct adhesion of the active portion of the drug product in contact with the oral mucosal membrane. In some instances, the bioadhesive may be at least somewhat permeable to the active pharmaceutical ingredient. The present disclosure includes both direct adhesion and/or indirect adhesion of the pharmaceutical formulation of active ingredient and at least one mucous membrane.

Some methods can also include sheathing the pharmaceutical formulation of the at least one active ingredient (e.g., l-epinephrine) with a barrier to protect from mixing with saliva outside of a zone/region where the pharmaceutical formulation of the at least one active ingredient is adhered to the at least one oral mucous membrane, to prevent the swallowing of medicated saliva. In these embodiments, the barrier at least partially comprises or is structurally and/or functionally associated with the at least one bioadhesive excipient.

In many embodiments, the bioadhesive excipient will be a bioadhesive polymer, which by definition is a synthetic or natural polymer which binds, sticks, or adheres to biological target tissue, namely in this case, mucosal membranes, at least temporarily. Mucosal membranes line externally exposed passageways of the body—such as the mouth, nose, respiratory tract, gastrointestinal tract, and vagina—with mucus, which helps protect and keep moist these surfaces. Embodiments of this disclosure are applied to the oral mucosal membranes, although application may be to other mucosal membranes. Mucus includes water, mineral salts, and the glycoprotein known as mucin. The sublingual gland under the tongue produces mucin. The bioadhesive can stick to these moist, often mucousy, epithelial tissue surfaces. In some embodiments the bioadhesive is a mucoadhesive, in which case, interaction and/or adhesion with mucus takes place.

For bioadhesion to occur, prolonged contact between the bioadhesive polymer(s) and the mucosa helps bind bioadhesive polymers to the mucin/epithelial surface. Generally, the bioadhesive polymer(s) will wet and swell over the mucosal site, such as from interaction with moisture or saliva on the mucosal surface. It is believed that bioadhesive polymer chains will interdiffuse and intermingle with the mucosal membrane, and in some instances penetrating the mucus, and forming bonds with the entangled glycoprotein chains of the mucosa thereof. Stickiness may be associated with swelling. Different forces maintain the adhesion, whether cohesive, through van der Waals force, hydrogen bonding, ionic or chemical bonding, or a combination thereof.

The properties of the bioadhesion can be controlled via the selection of the bioadhesive materials, such as selecting bioadhesive polymers with the desired concentration, flexibility, hydrophilicity, swelling, and surface tension, and size/molecular weight of the bioadhesive polymer chains. The bioadhesive or bioadhesive formulation can be solid at first, like a tablet or lozenge, a powder, or be semi-solid, like a gel, membrane, or film. In some embodiments, the bioadhesive formulation can be a liquid or viscous liquid which forms a gel or solid upon interaction with saliva. The bioadhesive or bioadhesive formulation is therefore selected from the class including powders, solids, tablets, lozenges, patches, semi-solids, gels, films, tapes, viscous liquids and liquids, bioadhesives that form a gel or solid, or any combination thereof. The bioadhesive is selected to interact at the proper pH of the oral mucosal environment. The bioadhesive is or becomes sticky after interacting with or absorbing fluid.

In some embodiments, an at least one bioadhesive excipient is selected from the class of natural bioadhesive polymers and/or the class of synthetic bioadhesive polymers, or analogue, derivative, or combinations thereof. Some bioadhesives can be thought of as biocompatible glues, gums, pastes, or gels. Examples of members of the class of natural bioadhesive polymers include acacia gum, alginic acid, gelatin, guar gum, pectin, sodium alginate, sodium hyaluronate, and tragacanth. Examples of members of the class of semi-synthetic and synthetic bioadhesive polymers include carbomers and other polyacrylics, carboxymethyl cellulose, hydroxypropyl cellulose, hydroxypropyl methylcellulose, polycarbophil, polyethylene glycol, polyvinyl alcohol, polyvinylpyrrolidone, and silicone polymers. An example of a bioadhesive paste includes gelatin, pectin, xanthan gum, polyethylene glycol, carboxymethylcellulose, mineral oil, and optionally, one or more parabens. This is different from an adhesive oral paste formulation used for many decades containing gelatin, pectin, and carboxymethylcellulose. An example of a bioadhesive film applied as a gel includes boric acid, hydroxypropylcellulose, propylene glycol, purified water, salicylic acid, denatured alcohol, and tannic acid. Lactose, acacia, and magnesium stearate can be used as a bioadhesive for a buccal tablet, and can be, for example, in a proportion, e.g., 10 mg, 30 mg, and 10 mg, although need not be in whole number ratios.

While the bioadhesive(s) holds the at least one active ingredient or pharmaceutical formulation of active ingredient(s) in place on the oral mucosa according to this alternative method, the active ingredient will be absorbed transmucosally either by passive diffusion, facilitated passive diffusion, active transport, pinocytosis, or a combination thereof. The sheathing of the at least one active ingredient or pharmaceutical formulation of active ingredient(s) protects against the swallowing of medicated saliva and from interfering with transmucosal absorption/delivery.

The bioadhesive layer or barrier may be hydrophilic on the side facing the mucosal surface, to absorb moisture from the mucus layer and become sticky. The bioadhesive layer or barrier may be hydrophobic and moisture-repellent on the opposite side, so as to prevent other saliva from entering the zone/region of the drug product and to prevent binding to mucosa surfaces on both sides. There can be some instances where the bioadhesive layer or barrier is hydrophilic on both sides, including when adhering to at least two mucosal surfaces simultaneously is desired. These instances will generally include adherence to the corner of the gingiva, where it interfaces with the cheek or floor of the mouth. It can also be desired to have the bioadhesive stick to the bottom tongue surface and the floor of the mouth simultaneously. When at least two mucosal surfaces are bound simultaneously by the same drug product dosage, it can be desirable for transmucosal delivery to also occur at the at least two mucosal surfaces simultaneously, for enhanced efficiency or quickness of transmucosal delivery. Adherence to at least two mucosal surfaces simultaneously can greatly increase the total adherence and/or total adherence surface area, and can help ensure that the drug product is secure and will not be swallowed during transmucosal delivery. A larger drug dose and/or extended release drug dose can be had with stronger/longer bioadhesion times. This can be helpful because treating hypotension from anaphylaxis may require multiple doses of epinephrine, and extended release may minimize the number of repeat doses.

It may be helpful for the bioadhesive to rapidly adhere to the mucosa, adhere adequately or strongly, and without interfering with drug release and transmucosal delivery. It may be helpful for the bioadhesive to not be influenced by the food and drink recently taken by the patient. If the patient's mouth is dry or wet, it may be helpful for the bioadhesive to bind well to the mucosa in either case.

It may also be helpful for the bioadhesive to work well with drug penetration enhancers, such as having a liposomal formulation (e.g., liposomal epinephrine), or have some drug penetration properties itself for transmucosal delivery, such as containing dimethyl sulfoxide (DMSO) solvent. The bioadhesive may facilitate contact of the active ingredient (e.g., epinephrine) with the underlying surface under the mucus layer. Again, the drug product may be secured and/or adhered directly and/or indirectly to the oral mucosal membrane by bioadhesive.

In embodiments with a bioadhesive, transmucosal delivery of the at least one active pharmaceutical ingredient including l-epinephrine, finishes well before bioadhesion stops, such as well before mucin turnover occurs. In these embodiments, the bioadhesive can be manually removed from the patient's oral mucosa after a period of time, for example after at least 1 minute or 2 minutes. In other embodiments, the bioadhesive is a natural or safe bioadhesive that can dissolve or be chewed and swallowed or spit out after the drug has been delivered.

A saliva barrier that is a bioadhesive/bioadhesive formulation or a saliva barrier with a bioadhesive/bioadhesive formulation is adjacent to or at least partially surrounding the pharmaceutical formulations of epinephrine for transmucosal delivery in some embodiments. The barrier may include polymers. The bioadhesive may include gums. The epinephrine formulation can therefore be associated with hydroxypropylcellulose, hydroxypropyl methylcellulose, polyvinylpyrrolidine and/or propylene glycol, and acacia or xanthan gum as barrier/bioadhesive. Other embodiments can include pregelatinised starch. The amounts of these barrier/bioadhesive materials/excipients can vary depending on the strength and longevity of the desired bioadhesive function, and are not meant to be limiting. Some embodiments can also include menthol, which may enable the user to feel the dosage on the mucosal surface working.

In other embodiments, epinephrine or a liquid formulation thereof is spray dried onto beads or another surface, such as the bottom surface of a tablet or physical barrier. In such embodiments, epinephrine or its formulation becomes a coat or coating layer to a surface or physical barrier. The disclosed methods may be carried out in further ways, and these examples are not meant to be limiting.

These methods can include at least two dosages of the pharmaceutical formulation of epinephrine. The at least two dosages of the pharmaceutical formulation of epinephrine can be in two different dosage amounts/concentrations of the pharmaceutical formulation of epinephrine.

When the method includes at least two different dosages/dosage amounts/dosage concentrations of the pharmaceutical formulation of epinephrine, each of the at least two different dosages/dosage amounts/dosage concentrations of the pharmaceutical formulation of epinephrine can be specifically calibrated for transmucosal delivery to a different mucous membrane inside an oral cavity. The mucosal surfaces inside a patient's mouth include, but are not limited to, sublingual, sublabial, buccal, palate, and gingiva surfaces. Dosage administration of the pharmaceutical formulation of epinephrine can be rotated among different mucosal surfaces inside the patient's mouth to prevent local complications of drug delivery to any one such the mucosal surface during a treatment regimen, the local complications of drug delivery including localized side effects or reduced diffusion/transport.

In some situations, such as when the mouth has been damaged, methods also include rectal administration in a human with an at least one dosage of a sublingual (or transmucosal) l-epinephrine formulation.

In some embodiments, the methods are practiced on animals, such as dogs and horses.

The transmucosal delivery is by passive diffusion across a mucous membrane and/or by active transport across a mucous membrane. In some embodiments, an electrical charge can be applied to the formulation via the applicator device to drive the transmucosal delivery.

The disclosed methods can provide temporary enhanced speed, physical strength, or physical endurance in the patient or user. These effects can have applications for the military, federal agents, law enforcement agents, first responders, and even athletes. Superhuman ability may be needed in emergency situations or to accomplish above normal feats.

The disclosed methods include holding and/or protecting an at least one oral transmucosal formulation with the tongue for over a minute. The methods include holding and/or protecting an at least one sublingual formulation under the tongue for over a minute. The methods include holding and/or protecting an at least one sublingual tablet formulation under the tongue for over a minute. The methods include holding and/or protecting an at least one l-epinephrine sublingual tablet formulation under the tongue for over a minute, or over 2 minutes or more. The methods include consecutive dosing of holding and/or protecting an at least one sublingual formulation under the tongue. The methods include consecutive dosing of holding and/or protecting an at least one l-epinephrine sublingual tablet under the tongue.

Intranasal, Nasal, and/or Inhalable Epinephrine

In embodiments, the present disclosure provides formulations of one or more active pharmaceutical ingredients and/or hemodynamic agents, such as l-epinephrine, for nasal administration, nasal or oral inhalation, or intranasal delivery, and methods of administering the same. In embodiments, the present disclosure provides inhalable formulations of one or more active pharmaceutical ingredients and/or hemodynamic agents, such as l-epinephrine, and methods of administering the same. In embodiments, the present disclosure provides formulations of one or more active pharmaceutical ingredients and/or hemodynamic agents, such as l-epinephrine, adapted for transmucosal absorption, and methods of administering the same.

Intranasal administration, as used herein, refers to the absorption of a pharmaceutical agent by the mucosa of the nasal cavity for systemic effects. The nasal cavity, as used herein, generally refers to the interior of the nose, and may extend from the nostrils to the pharynx.

Nasal administration, as used herein, relates to the dispensing or application of an agent into the nose and/or past the nose, such as inside the nasal cavity and/or further into the pharynx and/or further into the respiratory system, such as the trachea, bronchi, and deeper into the lungs.

Nasal administration can include the physical placing of a pharmaceutical agent into or up the nose with an applicator. Nasal administration can include nasal inhalation, in which a negative pressure generated by a patient draws or sucks the pharmaceutical agent into and/or past the nose. The negative pressure of nasal inhalation can be regulated by the patient's force of inhalation, by closing off one of the nostrils, or by how tightly a dispensing container or nasal applicator seals a nostril. Negative pressure may also be regulated from a delivery device by regulating airflow restriction, airflow velocity, or airflow path(s) and/or vent(s) of the device. Nasal administration can include insufflation, the blowing of a pharmaceutical agent into and/or past the nose using a positive pressure, such as from a pump, squeeze bottle, propellant, or using exhaled breath from the mouth routed into the nose. Nasal insufflation and nasal inhalation may be combined during nasal administration, such as by spraying a pharmaceutical agent into the nose while inhaling through the nose.

The agent administered via nasal administration may be at least one pharmaceutical agent or at least one pharmaceutical formulation including at least one pharmaceutical ingredient and at least one pharmaceutically acceptable excipient and/or propellant. In some embodiments, the pharmaceutical ingredient is an active pharmaceutical ingredient, such as a hemodynamic agent, which may be l-epinephrine. The active pharmaceutical ingredient or formulation may act locally or topically where it is applied, or may act systemically when absorbed into the bloodstream by transmucosal absorption, such as in the nasal cavity and/or alveolar regions of the lungs.

In embodiments, the at least one pharmaceutical agent for nasal administration may be formulated as a liquid, a suspension, a semi-viscous material, a gel, an emulsion, a liposome, an aerosol or aerosolizable substance, a dry material, such as a dry powder, or an encapsulated agent. The formulation may include a propellant. In embodiments, the at least one pharmaceutical agent may be adapted for transmucosal absorption. The at least one pharmaceutical agent may be provided in a capsule or may include an encapsulated dry powder formulation that opens or becomes unencapsulated or disaggregated before or during inhalation. The at least one pharmaceutical agent may be provided in a tablet that is crushed, or in a capsule or blister pack that is pierced and opened, before dispensing the pharmaceutical agent formulation.

In embodiments, the l-epinephrine formulation is inhalable, (i.e., capable of being inhaled, or sucked or drawn in), either by nasal inhalation and/or by oral inhalation. The inhalable l-epinephrine formulation may be formulated as a liquid, a suspension, a semi-viscous material, a gel, an emulsion, a liposome, an aerosol or aerosolizable substance, a dry material, such as a dry powder, or an encapsulated agent. The inhalable formulation may include a propellant. The inhalable l-epinephrine formulation may be provide in or as a capsule or may include an encapsulated dry powder formulation that opens or becomes unencapsulated or disaggregated before or during inhalation. The inhalable formulation may be provided in or as a tablet that is crushed, or a capsule or blister pack that is pierced and opened, before dispensing the l-epinephrine formulation.

In embodiments, the l-epinephrine formulation is adapted to be insufflated, such that is able to be pushed, squirted, or sprayed into and/or past the nose. In embodiments, the insufflatable l-epinephrine formulation may be formulated as a liquid, a suspension, a semi-viscous material, a gel, an emulsion, a liposome, an aerosol or aerosolizable substance, a dry material, such as a dry powder, or an encapsulated agent. The insufflatable formulation may include a propellant. In embodiments, the insufflatable l-epinephrine formulation may be adapted for transmucosal absorption.

In embodiments, a dispensing container or device includes a nasal inhaler, an intranasal inhaler, an oral inhaler, an oral inhaler with a nasal adapter, or an inhaler adapted for both nasal inhalation and oral inhalation. In some embodiments, a nasal prong or nostril fitting adapter having at least one inlet and at least one outlet may be placed over a generally larger outlet of an oral inhaler, such as a metered dose or pressurized meter dose inhaler, to enable nasal inhalation or oral inhalation. In some embodiments, a switch or valve may permit selection between a larger oral outlet and a smaller nasal outlet of the inhaler. While a given outlet may be designed or intended for nasal inhalation, the nasal outlet may also permit oral inhalation from the same.

A dispensing device that includes an oral inhaler with a nasal adapter or an inhaler adapted for both nasal inhalation and oral inhalation provides benefits over, for example, a device configured for oral inhalation that is instead used for nasal inhalation. When the oral outlet of a device configured for oral inhalation is placed under one or both nostrils, the outlet may not cover the nostril opening or openings well. Such anatomic incompatibility may lead to inefficient administration, loss of at least a portion of a dose, and/or low delivery of the inhalable pharmaceutical agent.

In some embodiments, both the oral outlet and nasal outlet dispense or administer the inhalable pharmaceutical agent simultaneously as the user inhales through the mouth and nose simultaneously. In some embodiments, the pharmaceutical agent is administered to or up both nostrils simultaneously, such as by using a nasal mask or a dispensing container having at least one outlet adapted to at least partially fit inside of both nostrils of the user simultaneously. In embodiments, a dispensing container or device may include a metered dose nasal spray applicator or metered dose nasal inhaler. The container or device may deliver a predetermined amount of pharmaceutical agent to the desired location, such as the lungs. In embodiments, a dispensing container may include a pressurized metered dose nasal inhaler (pMDI). Examples of propellants in a pMDI canister include hydrofluoroalkane (HFA) propellants. In embodiments, a dispensing container includes a dry powder nasal inhaler or dry powder nasal sprayer or nasal powder insufflator.

A dispensing device or nasal applicator or adapter thereof may be inserted at least partially inside at least one nostril of the user or patient, and the formulation may be dispensed up the nose, past the nostril. Transmucosal intranasal delivery of the formulation (e.g., an inhalable l-epinephrine formulation) may be accomplished by releasing the formulation into the nasal cavity for intranasal administration. Transmucosal intranasal administration and absorption inside the nasal cavity, such as by the nasal respiratory mucosa or epithelium, may help achieve systemic delivery of an agent, such as l-epinephrine, in the formulation. The agent may be absorbed by the capillary microvasculature in the mucosa. Transmucosal intranasal administration may also allow for central nervous system administration by absorption of a pharmaceutical agent or therapeutic substance from the olfactory epithelium, nerves, or olfactory lobe. Intranasal delivery can provide a non-invasive drug delivery route via various routes.

A nasal inhalation dose may be dispensed into or past at least one nostril for nasal inhalation or intranasal delivery. The dose may be administered to the nose, nasal cavity, and/or further into the respiratory system. The dose may be dispensed from a dispensing device as described above.

In some implementations, the l-epinephrine formulation may be administered by oral inhalation instead of, or in addition to, nasal administration. Oral inhalation may be helpful if a patient's nose or nostrils are blocked or damaged, or if a patient is suffering from respiratory distress or is having trouble breathing, such as from an allergic reaction or anaphylaxis. Oral inhalation may be helpful to prophylactically treat a patient who has been or may be exposed to an allergen, such as an insect sting or sulfite, before the onset of symptoms, such as anaphylaxis or bronchoconstriction. Oral inhalation may be helpful in patients experiencing bronchoconstriction, regardless of whether the patient is an asthmatic, to transiently treat or pretreat breathing difficulty, to temporarily stabilize the patient, and/or to relieve bronchoconstriction or anticipated bronchoconstriction. The relief of bronchoconstriction, even if temporary, may help the patient relax and more effectively and efficiently inhale a pharmaceutical agent into or through the nose. The administration of an oral inhalation dose of l-epinephrine to relieve bronchoconstriction and permit subsequent administration of a nasal inhalation dose of l-epinephrine, which subsequent administration may be more effective than in the absence of the oral inhalation dose, is believed to have not been previously practiced.

In embodiments, a dose administered by oral inhalation is lower (of lesser strength or amount) than a dose administered by nasal inhalation. If an oral inhalation dose is relatively large, e.g., 5 mg or more of l-epinephrine, and/or if the oral inhalation is not efficient, some of the l-epinephrine may be deposited at the back of the throat and be swallowed, which may cause gastrointestinal discomfort or side effects. In some embodiments, an oral inhalation dose is of equal or greater strength or amount than a nasal inhalation dose. In some embodiments, both an oral l-epinephrine inhalation dose and a nasal l-epinephrine inhalation dose are administered to a patient, and the oral inhalation dose is administered before the nasal inhalation dose is administered, and may be the first dose administered. In some embodiments the oral inhalation dose is administered after the nasal inhalation dose as a second, third, or more subsequent dose.

An oral inhalation dose may be dispensed into or past the patient's mouth for delivery to the patient's lungs. The dose may be administered to the upper and/or lower airways. Without being limited to any mechanism or mode of action, systemic absorption of l-epinephrine may occur in the alveolar region of the lungs. The dose may be delivered by a dispensing device as described above.

In some implementations, the l-epinephrine formulation by be administered buccally or sublingualy instead of, or in addition to, nasal administration or oral inhalation. In some embodiments, a capsule or tablet and/or encapsulated formulation (e.g., of l-epinephrine) is placed under the tongue or buccally against the cheek. In some embodiments, a pharmaceutical agent (e.g., l-epinephrine formulation) is delivered inside the mouth, such as under the tongue or buccally against the cheek, from a dispensing device. In some embodiments, the pharmaceutical agent (e.g., l-epinephrine formulation) is delivered inside the mouth by spraying such that the agent covers different regions of the mouth, which may enable oral transmucosal absorption. Administration to the interior of the mouth may be done purposefully, or may occur if oral inhalation is not performed well. Buccal or sublingual administration may be helpful when a patient, which may be a soldier, is losing consciousness or is otherwise unable to inhale a dose of a pharmaceutical agent. The bucally or subligually administered dose may provide enough of the pharmaceutical agent (e.g., l-epinephrine formulation) to the patient such that the patient regains mental focus or consciousness. The next dose may be administered intranasally or by oral inhalation.

In some embodiments, two or more dosages of the pharmaceutical agent (e.g., an l-epinephrine formulation) may be administered. The first dosage and the second dosage may include the same amount of pharmaceutical agent, or may include different amounts of pharmaceutical agent. In some embodiments, the amount of agent delivered to a patient is adjustable (or selectable or titratable) for at least one of the first or second dosages. An l-epinephrine formulation, which may be an inhalable l-epinephrine formulation, may include at least one excipient ingredient, at least one additional active pharmaceutical ingredient, and/or at least one therapeutic substance. The at least one additional active pharmaceutical ingredient is not limited to any class of active pharmaceutical ingredients. As non-limiting examples, the at least one additional active pharmaceutical ingredient may be a stimulant, an antihistamine (e.g., diphenhydramine), a bronchodilator and/or short-acting beta-agonist (e.g., albuterol, levalbuterol, metaproterenol, pirbuterol, or terbutaline), an expectorant, an analgesic, an anticholinergic, a relaxant, or a corticosteroid (e.g., budesonide). The at least one therapeutic substance, may be naturally occurring or naturally derived, such as a cannabinoid or plant-based material or plant material (e.g., hemp or marijuana material).

The formulations described herein may include l-epinephrine and may also include additional components suitable for intranasal administration. The l-epinephrine may include, for example, l-epinephrine base or l-epinephrine salt, such as a bitartrate salt. In some embodiments, the formulations may be free of an epinephrine tartrate or bitartrate.

The l-epinephrine may be provided in an aqueous formulation suitable for intranasal administration. The aqueous formulation may include one or more of water or an alcohol, such as ethanol. The water and alcohol may be provided in any ratio. Examples include water:alcohol ratios of about 100:0 to about 0:100, about 90:10 to about 0:100, about 80:20 to about 0:100, about 70:30 to about 0:100, about 60:40 to about 0:100, about 50:50 to about 0:100, about 100:0 to about 10:90, about 100:0 to about 20:80, about 100:0 to about 30:70, about 100:0 to about 40:60, about 10:0 to about 50:50, or about 60:40 to about 40:60.

The aqueous formulation may include one or more acids. The one or more acids may help adjust the formulation to a desired pH. Examples of acids include hydrochloric acid, tartaric acid, citric acid, and sorbic acid. Acids may be provided at any concentration. In one example, 1N hydrochloric acid is included.

The aqueous formulation may include one or more bases. The one or more acids may help adjust the formulation to a desired pH. Examples of bases include sodium hydroxide and sodium phosphate. Bases may be provided at any concentration. In one example, 0.2N sodium hydroxide is included.

The aqueous formulation may include one or more salts. Examples of salts include sodium chloride, edetate disodium, potassium chloride, and sodium citrate. The one or more salts may help to buffer the formulation, adjust the tonicity of the formulation, and/or adjust the osmolarity of the formulation. The tonicity may be adjusted to a desired tonicity. A desired tonicity may be one that enhances intranasal transmucosal delivery while minimizing discomfort of delivery to the nasal cavity. The tonicity of the formulation may be isotonic, hypotonic, or hypertonic. The osmolality may be adjusted to a desired osmolality. A desired osmolality may be between about 290 mosm/kg and about 500 mosm/kg.

The aqueous formulation may include one or more antioxidants or preservatives, such as sodium metabisulfite, tocopherols, edetate disodium, chlorobutanol, potassium sorbate, benzalkonium chloride, sodium benzoate, ethanol or other alcohols, and methyl paraben.

The aqueous formulation may include one or more excipients that help increase viscosity, such as glycerin, propylene glycol, or polyethylene glycol.

The aqueous formulation may include one or more absorption enhancer or penetration enhancer. An absorption enhancer/penetration enhancer may help increase absorption speed. An absorption enhancer/penetration enhancer may enable a reduction in the amount of epinephrine in a given dosage. Examples of absorption/penetration enhancers include medium chain fatty acids, their salts, and derivatives thereof. Linoleic acid is one example of a suitable fatty acid. Other examples of absorption/penetration enhancers include DMSO, farnesol, alkylglycosides, and liposomes or lipid micelles/lipid polymer micelles. Penetration enhancers may also include solvents such as ethanol, propylene glycol, diethylene glycol monoethyl ether, and/or oleic acid or other fatty acids; terpenes, such as menthol, camphor, and methyl salicylate; and surfactants, such as tween 80, N-methyl-pyrrolidone, benzalkonium chloride, castor oil, and didecyldimethylammonium bromide; and any combinations and/or related compounds, salts, or derivatives thereof.

The formulation may include a sugar, which may help adjust tonicity. In some embodiments, the formulation may include a surfactant such as polysorbate. In some embodiments, the formulation may include a suspending agent such as carboxymethyl cellulose.

The aqueous formulation may be free of sulfites, such as free of metabisulfite. Some individuals are sensitive to sulfite and sulfite exposure can trigger an allergic reaction or even anaphylaxis. Treating anaphylaxis, for example, in a sulfite-sensitive individual with a sulfite-containing formulation can in turn trigger anaphylaxis. A formulation free of sulfites may help reduce or eliminate a secondary allergic reaction or side effect in sulfite-susceptible individuals.

The aqueous formulation may be free of chitin. Chitin is derived from shellfish and can be an allergen. A formulation free of chitin may reduce or eliminate an allergic reaction in susceptible individuals.

L-epinephrine may be present in an aqueous formulation at any concentration. In examples, l-epinephrine is present at a concentration of greater than about 1 mg/mL. The l-epinephrine may be present at a concentration of about 1.5 mg/mL to about 19 mg/mL, about 1.5 mg/mL to about 12 mg/mL, about 2 mg/mL to about 12 mg/mL, about 3 mg/mL to about 12 mg/mL, about 4 mg/mL to about 12 mg/mL, about 5 mg/mL to about 12 mg/mL, about 6 mg/mL to about 12 mg/mL, about 7 mg/mL to about 12 mg/mL, about 8 mg/mL to about 12 mg/mL, about 1.5 mg/mL to about 11 mg/mL, about 1.5 mg/mL to about 10 mg/mL, about 1.5 mg/mL to about 9 mg/mL, about 1.5 mg/mL to about 8 mg/mL, about 1.5 mg/mL to about 7 mg/mL, about 1.5 mg/mL to about 6 mg/mL, about 1.5 mg/mL to about 5 mg/mL, about 1.5 mg/mL to about 4 mg/mL, or about 6 mg/mL to about 10 mg/mL.

The concentration of l-epinephrine in an aqueous formulation may be provided as a weight/weight percentage. In examples, l-epinephrine is present at a concentration of about 0.5% w/w to about 7% w/w, about 1% w/w to about 7% w/w, about 1.5% w/w to about 7% w/w, about 2% w/w to about 7% w/w, about 2.5% w/w to about 7% w/w, about 3% w/w to about 7% w/w, about 3.5% w/w to about 7% w/w, about 4% w/w to about 7% w/w, about 0.5% w/w to about 6.5% w/w, about 0.5% w/w to about 6% w/w, about 0.5% w/w to about 5.5% w/w, about 0.5% w/w to about 5% w/w, about 0.5% w/w to about 4.5% w/w, about 0.5% w/w to about 4% w/w, or about 0.6% w/w to about 6% w/w.

The one or more acids may be present in an aqueous formulation at about 24% w/w to about 36% w/w, about 26% w/w to about 36% w/w, about 28% w/w to about 36% w/w, about 30% w/w to about 36% w/w, about 24% w/w to about 34% w/w, about 24% w/w to about 32% w/w, about 24% w/w to about 30% w/w, or about 28% w/w to about 32% w/w. In some examples, the one or more acid is present in an amount sufficient to adjust the pH of the aqueous formulation to a desired pH.

The desired pH may be one that is compatible with the intranasal mucosa and/or intranasal delivery. The desired pH may be greater than 1. The desired pH may be about 3 to about 7.5, about 3.5 to about 7.5, about 4 to about 7.5, about 4.5 to about 7.5, about 5 to about 7.5, about 5.5 to about 7.5, about 3 to about 7, about 3 to about 6.5, about 3 to about 6, about 3 to about 5.5, about 3 to about 5.5, about 3 to about 4.5, or about 3.5 to about 5.

The one or more bases may be present in an amount sufficient to adjust the pH of the aqueous formulation to a desired pH.

The one or more salts may be present in an aqueous formulation at any concentration. In one example, the concentration of salt may approximate that of saline (about 9 mg/mL).

The one or more antioxidants or preservatives may be present in an aqueous formulation at about 0.25% w/w to about 0.5% w/w, about 0.3% w/w to about 0.5% w/w, about 0.35% w/w to about 0.5% w/w, about 0.4% w/w to about 0.5% w/w, about 0.25% w/w to about 0.45% w/w, about 0.25% w/w to about 0.4% w/w, or about 0.25% w/w to about 0.35% w/w.

The one or more excipients may be present in an aqueous formulation at about 4% w/w to 6% w/w, about 4.5% w/w to 6% w/w, about 5% w/w to 6% w/w, about 4% w/w to 5.5% w/w, or about 4% w/w to 5% w/w.

The one or more absorption enhancer or penetration enhancer may be present in an aqueous formulation at about 1% w/w to about 40% w/w, about 5% w/w to about 40% w/w, about 10% w/w to about 40% w/w, about 15% w/w to about 40% w/w, about 20% w/w to about 40% w/w, about 25% w/w to about 40% w/w, about 30% w/w to about 40% w/w, about 1% w/w to about 35% w/w, about 1% w/w to about 30% w/w, about 1% w/w to about 25% w/w, about 1% w/w to about 20% w/w, about 1% w/w to about 15% w/w, or about 1% w/w to about 10% w/w.

In one example, an aqueous formulation includes the following components: epinephrine at about 0.6% w/w to about 6% w/w, a 1N solution of hydrochloric acid at about 24% w/w to about 36% w/w, sodium metabisulfite at about 0.25% w/w to about 0.5% w/w, ethanol (absolute) or dehydrated alcohol at about 32% w/w to 48% w/w, and propylene glycol at about 4% w/w to 6% w/w. Water may also be included to 100%, which may be about 18% w/w to about 24% w/w. Sodium hydroxide, such as a 0.2N solution, may be used to adjust the pH to about 3 to about 4.5.

In one example, an aqueous formulation includes the following components: epinephrine at about 6% w/w, a 1N solution of hydrochloric acid at about 30% w/w, sodium metabisulfite at about 0.3% w/w, ethanol at about 40% w/w, propylene glycol at about 5% w/w, and water at about 18% w/w. Sodium hydroxide, such as a 0.2N solution, may be used to adjust the pH to about 4.5.

In some embodiments, the intranasal l-epinephrine formulation is shelf stable. For example, the intranasal l-epinephrine formulation may include less than 6% d-epinephrine impurity at release. The intranasal l-epinephrine formulation may include less than 12% d-epinephrine impurity over a shelf-life of at least 12 months. In some embodiments, the intranasal l-epinephrine formulation may include higher amounts of d-epinephrine at release or after 12 months or both. For example, formulations may include up to about 20%, about 30%, about 40%, or about 50% d-epinephrine.

The l-epinephrine formulation for intranasal administration may be packaged in or administered from a dispensing container. The dispensing container may be configured to release the l-epinephrine formulation into or past either or both of a recipient's nostrils for transmucosal intranasal delivery inside the nasal cavity. The dispensing container may be, for example, a nasal spray applicator, such as an atomizing or non-atomizing nasal spray applicator; a squeeze bottle; a metered dose nasal spray applicator; a pressurized metered dose nasal spray applicator; a nasal spray applicator including a spray pump/pump mechanism; a nasal spray applicator having at least one outlet or nozzle adapted to at least partially fit inside of one nostril or both nostrils simultaneously; a syringe having an outlet or nozzle adapted to at least partially fit inside of one nostril or both nostrils simultaneously; and a syringe having a nasal spray atomizing nozzle adapter, which may be connected to the syringe by a luer fitting. Packaging the l-epinephrine formulation in a nasal spray applicator may provide benefits including easy administration, including into the nasal cavity, and convenient transport. Packaging the l-epinephrine formulation in a syringe may provide benefits including the ability to administer the contents by other than intranasal administration, such as by injecting the contents into an IV bag for intravenous administration.

In some methods of using a dispensing container disclosed above, the luer fitting may be inserted directly into a nostril of a patient. In other methods, the dispensing container is configured to spray atomized droplets and/or aerosolized particles of the l-epinephrine formulation into, or past, at least one of nostril of a patient for intranasal delivery. An atomized spray for intranasal delivery may include a mist of droplets having a size distribution ranging from about 20 micrometers to about 100 micrometers in diameter. In embodiments, a spray may include droplets sized less than 20 micrometers in diameter or less than 10 micrometers in diameter. The droplets may be small enough to be inhalable into the lungs.

A dispensing container may hold one or more dosages of an l-epinephrine formulation. A dosage may have a volume of about 0.05 mL to about 1 mL, about 0.1 mL to about 1 mL, about 0.3 mL to about 1 mL, about 0.5 mL to about 1 mL, about 0.7 mL to about 1 mL, about 0.05 mL to about 0.8 mL, about 0.05 mL to about 0.6 mL, about 0.05 mL to about 0.4 mL, about 0.05 mL to about 0.2 mL, about 0.1 mL to about 0.6 mL, about 0.15 mL to about 0.35 mL, or about 0.05 mL to about 0.35 mL. Higher volumes may drip down from the nasal cavity into the pharynx/oropharynx. In some implementations, dripping into the pharynx/oropharynx is not desirable because the nasal cavity is the target for administration. In some implementations, dripping into the pharynx/oropharynx is desirable because it increases the surface area available for transmucosal absorption, and may approach the trachea and/or help open constricted airways.

A dosage of an l-epinephrine formulation may include about 0.1 mg to about 50 mg of l-epinephrine, about 1 mg to about 50 mg, about 5 mg to about 50 mg, about 10 mg to about 50 mg, about 20 mg to about 50 mg, about 30 mg to about 50 mg, about 0.1 mg to about 40 mg, about 0.1 mg to about 30 mg, about 0.1 mg to about 20 mg, about 0.1 mg to about 10 mg, about 0.1 mg to about 5 mg, about 0.1 mg to about 1 mg, about 1 mg to about 15 mg, or about 2 mg to about 8 mg of l-epinephrine.

In embodiments, formulations include pure or substantially pure l-epinephrine. The formulations may be free or substantially free of impurities. The l-epinephrine may have good stability. The formulations may be stable with little to no racemization to d-epinephrine. In some embodiments, the formulations do not include an alcohol, such as ethanol. In other embodiments, the formulations include an alcohol, such as ethanol. The formulations may include a mucoadhesive and/or absorption enhancing excipient.

The formulations may be inhalable and/or adapted for transmucosal delivery. The formulations may be in the form of a nasal spray or in the form of a pressurized metered dose. An example of a nasal dose is greater than 7 mg of l-epinephrine, which may be divided between nostrils. The formulation may be provided as a pMDI formulation. The pMDI formulation may be provided in a pMDI nasal inhaler or in an oral inhaler, which may include a removable nasal outlet. The pMDI formulation may include about 3 mg of epinephrine to about 20 mg of epinephrine, such as about 9 mg to about 12 mg of epinephrine. The pMDI formulation may include one or more of a propellant (e.g., HFA-134a, compressed air, or compressed gas(es) such as oxygen and/or nitrogen), ethanol, thymol (a monoterpenoid phenol that may function as a preservative and/or as an antiseptic), polysorbate 80 (a nonionic surfactant and emulsifier), oleic acid, and norflurane, and optionally an alcohol. A nasal pMDI inhaler may include at least two dosages to be administered to a user. Dosages can be divided among the nostrils and/or between the oral inhalation and nasal inhalation routes and/or sublingual and buccal routes. Any one or more of the dosages may be adjustable, such as being adjustable in amount to provide a nasal inhalation dose or intranasal dose greater than an oral inhalation dose.

In embodiments, the presently disclosed formulations may include a dry form of a pharmaceutical agent, such as l-epinephrine. Formulations may include only a pure dry, such as powdered, pharmaceutical agent (e.g., pure l-epinephrine API powder), which may be dispensed or snorted into the nose. Formulations may also include a carrier and/or bulking agent excipient in an amount conducive to nasal and/or oral inhalation. A carrier and/or bulking agent may be a sugar, such as a disaccharide, such as lactose, lactose monohydrate, or its salt or derivate thereof. Formulations may include a propellant. A lubricant and/or releasing agent, such as magnesium stearate, may be included in some formulations. A mucoadhesive and/or absorption enhancing excipient may be included in some formulation. Formulations may include other excipients or classes of excipients, such as sodium citrate, mannitol, glycine, and sodium hydroxide. A dose of a dry powder formulation may include more than about 3 mg of l-epinephrine, such as about 7 mg to about 12 mg. The l-epinephrine can be divided among the nostrils and/or between oral inhalation and nasal inhalation routes and/or sublingual and buccal routes.

Compared to known formulations of inhalable epinephrine, the presently disclosed formulations may include pure or substantially pure l-epinephrine (e.g., formulations including less than 6% d-epinephrine impurity at release and/or less than 12% d-epinephrine impurity over a shelf-life of at least 12 months) instead of racepinephrine (i.e., a racemic mixture of l-epinephrine and d-epinephrine). Compared to known formulations of inhalable epinephrine, which may include 0.125 mg of epinephrine (Primatene® Mist, Amphastar Pharmaceuticals, Inc.), the presently disclosed formulations may include up to about 150 times, such as about 25 times to about 75 times or about 50 times, the 0.125 mg amount. Compared to known devices for dispensing inhalable epinephrine, the presently disclosed formulations may be provided in a nasal pMDI inhaler instead of an oral inhaler. Other known devices are nebulizer jars in which liquid formulations are nebulized/aerosolized for oral inhalation.

In some embodiments, formulations of l-epinephrine are formulated for immediate release, which may provide rapid transmucosal delivery of an active l-epinephrine pharmaceutical agent. In some embodiments, formulations of l-epinephrine are formulated for extended or sustained release of an active l-epinephrine pharmaceutical agent. Formulations may include up to 1,000 mg or more of l-epinephrine, which may mimic intravenous infusion of epinephrine when slowly released. Such extended or sustained release formulations may at least partially substitute for continuous intravenous infusion. Such slow or controlled release formulations may be used to treat, for example, hypotension associated with septic shock or disease states associated with adrenal insufficiency. A mucoadhesive may be used and may affect transmucosal residence/clearance time and/or absorption.

Methods of Administering or Using Intranasal, Nasal, and/or Inhalable Epinephrine

Methods of administering intranasal formulations of hemodynamic, adrenergic, and/or vasoconstricting agents are also provided herein. While reference is made to intranasal l-epinephrine formulations, the methods may also apply to other agents such as norepinephrine, vasopressin, angiotensin II, and insulin or an analogue, derivative, mimetic, or prodrug thereof.

The methods are directed to non-intestinal transmucosal delivery of the agent, such as delivery via the intranasal mucosa. The transmucosal delivery is by passive diffusion across a mucous membrane and/or by active transport across a mucous membrane. In some embodiments, an electrical charge can be applied to the formulation to drive the transmucosal delivery.

Compared to other delivery methods, delivery via the intranasal mucosa may improve consistency of dosing, improve reproducibility of dosing, decrease suboptimal dosing, increase bioavailability, increase efficacy, increase drug transportation to the circulatory system, reduce metabolic and/or enzymatic degradation, and/or reduce side effects. Delivery via the intranasal mucosa may allow for easy self-administration as well as easy administration to someone else, who may be distressed or incapacitated. In one example, a parent, friend, school nurse, or first responder may be able to easily administer intranasal epinephrine as disclosed herein to a child suffering from a severe allergic reaction, anaphylaxis, or anaphylactic shock.

The disclosed methods may help restore blood perfusion, such as in an emergency situation; elevate plasma epinephrine levels; mimic epinephrine plasma pharmacokinetic parameters of an at least one injection of an injectable liquid l-epinephrine formulation, temporarily and/or for an extended period of time; and/or mimic plasma epinephrine levels of an at least one injection of an injectable liquid l-epinephrine formulation, temporarily and/or for an extended period of time. The methods may be practiced on a human or other animal, such as a dog or horse. The methods may meet an unfilled medical need in a safe, reliable, and comfortable, such as pain-free, manner. The disclosed methods may provide temporary enhanced speed, physical strength, or physical endurance in the patient or user. These effects can have applications for the military, federal agents, law enforcement agents, first responders, and even athletes. Superhuman ability may be needed in emergency situations or to accomplish above normal feats.

In any method of administrating intranasal l-epinephrine formulations, the patient's vitals may be monitored. Another individual may check the patient's breathing and pulse rate. If no one else is around, and if possible, the patient should check his or her own pulse rate. The term ‘patient’ is used, but the recipient of the intranasal l-epinephrine formulations can be anyone (e.g., soldier, policeman, fireman, first responder, student, outdoorsman/hiker, sailor, truck driver, and so on). The patient or individual receiving the intranasal formulations may have his/her head positioned upright, although the intranasal formulations can be administered in a supine position, especially if self-administered.

Methods of administering intranasal l-epinephrine formulations may include administering at least one dosage of the disclosed intranasal l-epinephrine formulations to a patient's nose or nostrils. The dosage may be administered by, for example, spraying the dosage into the nose or at least one nostril.

A patient may inhale before, during, or after administration, which may help increase transmucosal absorption of the dosage.

The dosage may be administered from any dispensing container described above. The container, or a portion thereof, may be inserted partially or fully into one or both nostrils.

Additional dosages, such as second and third dosages, may be administered similarly to the first dosage. Consecutive doses may be spaced apart by about 1 minute, at least about 5 minutes, at least about 10 minutes, or at least about 15 minutes. In some applications, consecutive dosages are administered to alternating nostrils. Alternating nostrils may help reduce or avoid local complications, such as localized toxicity or reduced transport at a site during a treatment regimen. Alternating nostrils may help maintain efficient drug transport and/or maximize bioavailability over a treatment regimen.

Each dosage may be the same or different. For example, the amount of l-epinephrine in each dosage may be the same or different. As another example, the volume of each dosage may be the same or different. As a further example, the release characteristics of each dosage may be the same or different. Multiple dosages may be provided from the same or different dispensing containers.

In some embodiments, a patient's nose or nostrils are blocked or damaged prior to administration of an intranasal l-epinephrine formulations. In some embodiments, a patient's nose or nostrils become blocked or damaged during administration of consecutive dosages of intranasal l-epinephrine formulation(s). The l-epinephrine formulations formulated for intranasal administration may be administered to another cavity when the formulation cannot or should not be administered to the nose or nostrils. For example, l-epinephrine formulations formulated for intranasal administration may be administered sublingually or buccally inside the oral cavity. As another example, l-epinephrine formulations formulated for intranasal administration may be administered to the trachea by tracheal intubation. Administration to a site other than the nasal cavity may be instead of or in addition to intranasal administration.

Compared to known methods of providing epinephrine, the presently disclosed methods may include providing each of a nasally administered dose and an orally administered dose in one treatment. Compared to known methods of providing epinephrine, the presently disclosed methods may include providing each of a nasally administered dose and a sublingually administered dose in one treatment. Administering epinephrine via more than one route in a single treatment may increase the surface area for drug absorption (e.g., nasal cavity in addition to lung tissue, nasal cavity in addition to sublingual tissue). Increasing the surface area available for drug absorption may decrease the total amount of drug needed to achieve a desired response, may decrease the amount of time to achieve a desired response, and/or may increase the duration of the drug's effectiveness. Compared to known methods of providing epinephrine, the presently disclosed methods may beneficially permit the administration of epinephrine via an alternative route, such as an oral route, when the intranasal route is not available.

Methods of administering the intranasal l-epinephrine formulations disclosed herein, to any site, may help elevate plasma epinephrine levels in a patient. Plasma epinephrine levels may be elevated to, and/or maintained at, at least about 40 pg/mL in a human patient. Maintenance may last for at least about 30 minutes. Administration of at least two dosages of the intranasal l-epinephrine formulations may better elevate or maintain plasma epinephrine levels compared to a single dosage.

Methods of administering the intranasal l-epinephrine formulations disclosed herein, to any site, may help maintain a constant or near constant raise in blood pressure in a patient. Maintenance may last for at least about 30 minutes. Administration of at least two dosages of the intranasal l-epinephrine formulations may better maintain a raise in blood pressure compared to a single dosage. The disclosed methods may normalize, or nearly normalize, a hypotensive state to a normotensive blood pressure. Blood pressure may be increased by a few mmHg, which may help reduce the loss of perfusion.

In addition to, or instead of, maintaining blood pressure, methods of administering the intranasal l-epinephrine formulations disclosed herein may help maintain at least one of pulse and breathing in a patient. The patient may be experiencing at least one of anaphylaxis, anaphylactic shock, sepsis, septic shock, respiratory difficulty, and cardiac difficulty.

In some embodiments, administration of at least one dosage of an at least one intranasal l-epinephrine formulation mimics epinephrine plasma pharmacokinetic parameters and/or plasma epinephrine levels of an at least one injection of an at least one injectable liquid l-epinephrine formulation. At least two dosages of the at least one intranasal l-epinephrine formulation, which may be administered at least 5 minutes apart, may better mimic the results of injecting an at least one injection of an at least one injectable liquid l-epinephrine formulation than does a single dosage.

The at least one injectable liquid l-epinephrine formulation may be at a concentration of 0.1 mg per mL to 1 mg per mL prior to any diluting. The at least one injectable liquid l-epinephrine formulation may contain 0.1 mg to 1 mg of l-epinephrine and/or the at least one injectable liquid l-epinephrine formulation may contain 0.1 mL to 10 mL of l-epinephrine prior to any diluting. The at least one injection of an at least one injectable liquid l-epinephrine formulation may be selected from endotracheal injection, intracardiac injection, intramuscular injection, subcutaneous injection, subcutaneous infusion, intravenous injection, and intravenous infusion.

Such injectable liquid l-epinephrine formulations are administered to treat individuals and/or patients who are experiencing any one or more of the following: hypotension, such as may occur from drug or allergen hypersensitivity reactions; anaphylaxis; anaphylactic shock; sepsis; septic shock; respiratory distress, such as may occur by bronchospasm, asthma/asthma attack, or respiratory infection; inadequate heart rhythm, such as may occur in cardiac arrest, arrhythmia, syncope/heart block, and those needing resuscitation; and mucosal congestion, such as may occur from allergy or sinus infection. In some instances, such injectable liquid l-epinephrine formulations can also be administered to prophylactically treat individuals and/or patients who are experiencing any of the above indications. The disclosed methods may treat any one or more of the above indications. The disclosed methods, using the disclosed intranasal l-epinephrine formulations, may result in similar physiologic (pharmacodynamic) effects as does an injection of l-epinephrine.

The disclosure includes a method of repeat dosing of an at least one intranasal l-epinephrine formulation to elevate plasma epinephrine levels to maintain at least one of blood pressure, pulse, and breathing in a human patient having at least one of severe allergy, anaphylaxis, anaphylactic shock, sepsis, septic shock, respiratory difficulty, and cardiac difficulty, when an at least one injection of an injectable liquid l-epinephrine formulation is not available and/or not possible, at least until emergency medical services arrive to treat and/or transport the human patient. The human patient may be a soldier on a battlefield or in a location without emergency medical services. The at least one injection of an injectable liquid l-epinephrine formulation may be administered later if available and possible.

The disclosure also provides a method of elevating plasma epinephrine levels, and/or a method of mimicking epinephrine plasma pharmacokinetic parameters and/or plasma epinephrine levels of an at least one injection of an injectable liquid l-epinephrine formulation, temporarily and/or for an extended period of time (e.g., a half hour), in a military soldier and/or military pilot with an at least one dosage of an intranasal l-epinephrine formulation. The disclosed methods and at least one dosage of an intranasal l-epinephrine formulation can be used to not only treat a soldier experiencing at least one of hypotension, respiratory difficulty, and/or cardiac difficulty, but also to enhance the physical and/or mental training of soldiers, and also to stimulate soldiers during extreme combat situations.

Soldiers, like other individuals, may be sensitive to food allergens and insect bites, which can trigger hypersensitivity reactions or anaphylaxis in susceptible individuals. The present disclosure reduces or negates the need for soldiers to carry around bulky and heat-sensitive injectable liquid formulations of epinephrine, whether as an epinephrine autoinjector or epinephrine syringes, vials, or ampoules. Instead, a small container, such as an inhaler, of one or more intranasal l-epinephrine formulations may be carried.

Compared to most individuals, soldiers are at greater risk of being exposed to many other chemical agents that may trigger anaphylaxis, whether it be from smoke inhalation of the burning of noxious materials or even from chemical warfare agents. The ATROPEN® is a prefilled auto-injector of Atropine Injection, an anticholinergic agent used to treat poisoning by susceptible organophosphorous nerve agents having cholinesterase activity as well as organophosphorous or carbamate insecticides. Atropine injection is believed to be part of an antidote kit prepared for the military. Likewise, intranasal l-epinephrine formulations could be included in an antidote kit for soldiers. The disclosed methods include the sequential dosing and/or co-administration of an at least one intranasal l-epinephrine formulation and an at least one atropine and/or hyoscyamine formulation, or other anticholinergic or antidote agent(s). The atropine (or its levorotary isomer hyoscyamine) may be administered as a liquid injection and/or as a solid oral formulation, and may be an intranasal formulation. In another embodiment, a combination intranasal formulation is administered containing both l-epinephrine active ingredient and atropine (and/or hyoscyamine) active ingredient as an antidote to nerve poisoning agent(s), or containing other anticholinergic or antidote agent(s). These examples are not meant to be limiting.

The disclosed intranasal l-epinephrine formulation or dosage form and methods can also be used to enhance the physical and/or mental training of combat soldiers and even combat vehicle drivers or fighter pilots by boosting blood perfusion throughout the brain and body with higher cardiac output, as well as increasing brain metabolism and oxygen consumption by the brain. The disclosed formulations, dosages, and methods may also simulate an ‘adrenaline rush’ during training that a soldier or pilot would otherwise experience out in the field. This may beforehand demonstrate the soldier's or pilot's ability to operate under the mental pressure and physical stress of combat or tactical missions.

Additionally, the disclosed intranasal l-epinephrine formulation or dosage form and methods can be used to stimulate soldiers and/or pilots during extreme combat situations. Such stimulation is believed to reduce reaction time and reduce fear, and even temporarily reduce pain, such as from a wound (e.g., bullet wound). This may be useful if a soldier charges several combatants in a high-risk situation or is in an intense dog fight with multiple combatants. With these methods, a soldier may impose maximum damage on the enemy. These methods may provide at least some chance of survival in what would otherwise be a ‘suicide mission’. The disclosed methods may also include the sequential dosing and/or co-administration of an at least one intranasal l-epinephrine formulation and the administration of an at least one stimulant and/or analgesic. The at least one stimulant and/or analgesic may be administered as liquid injection(s), topical/transdermal application(s), and/or solid oral formulation(s), or as intranasal or sublingual formulation(s). In another embodiment, a combination intranasal formulation is administered containing both l-epinephrine active ingredient and stimulant active ingredient (e.g., an amphetamine, e.g., a xanthine [caffeine or cocaine analogue]) and/or analgesic active ingredient (e.g., a member of the opioid class, an opioid, such as morphine or fentanyl, or another opioid analogue, derivative, or a combination thereof) to enhance the ability of the soldier and/or pilot during a combat situation. These examples are not meant to be limiting and other members of stimulant and/or analgesic classes can be substituted or additionally included.

The disclosed intranasal l-epinephrine formulation or dosage form and methods can also be used to prevent, delay, or reduce the time that a combat pilot, or even astronaut, may lose consciousness (i.e., blackout) from high acceleration or gravitational force (e.g., more than a several-fold g-force or a maximum g-force).

Still further, the disclosed intranasal l-epinephrine formulation or dosage form and methods can help revive a soldier after experiencing physical trauma or sedation.

The disclosure also provides a method of elevating plasma epinephrine levels, and/or a method of mimicking epinephrine plasma pharmacokinetic parameters/plasma epinephrine levels of an at least one injection of an injectable liquid l-epinephrine formulation, temporarily and/or for an extended period of time (e.g., a half hour), in an athlete with an at least one dosage of an intranasal l-epinephrine formulation. These methods can help revive an athlete experiencing exercise induced hypotension, bronchospasm, asthmatic episode or cardiovascular collapse. Further, the disclosed intranasal l-epinephrine formulation or dosage form and methods can also be used to enhance the physical and/or mental training of athletes by boosting blood perfusion throughout the brain and body with higher cardiac output, as well as increasing brain metabolism and oxygen consumption by the brain. The disclosed formulations and methods may also simulate an ‘adrenaline rush’ during athletic training that an athlete would otherwise experience out in the field during a game, race, or other athletic event or competition. Where permitted, when not considered ‘doping’, the disclosed intranasal l-epinephrine formulation or dosage form and methods can also be used to stimulate (professional) athletes or Olympians during an event to enhance performance, reduce reaction time and reduce fear, and even temporarily reduce pain. Epinephrine may increase blood flow to muscles and increase blood glucose levels, along with cardiac output, which may enhance athletic performance.

Further, the disclosure also provides a method of elevating plasma epinephrine levels, and/or a method of mimicking epinephrine plasma pharmacokinetic parameters/plasma epinephrine levels of an at least one injection of an injectable liquid l-epinephrine formulation, temporarily and/or for an extended period of time (e.g., a half hour), for neurocognitive enhancement, especially if combined with a ‘smart drug,’ ‘smart supplement’, or other cognitive enhancer (a ‘nootropic’ agent). Such nootropic agents may include supplements such as ginseng and Ginkgo biloba, and drugs such as racetams, e.g., piracetam and its derivatives. Nootropic agents can also include amphetamines (like Adderall® and Ritalin®), methylphenidate, modafinil, nicotine and their analogues, derivatives, and combinations thereof. These examples are not meant to be limiting. At least temporary neuroenhancement with the disclosed methods may help in thinking of better strategies and solutions to problems in science, medicine, business, warfare, and help ‘think tanks’ and innovation as well. These methods can enhance blood flow and oxygen utilization by the brain and stimulate synaptic activity.

The disclosed methods even allow for a ‘cardiac stress test in a bottle’, as a method of simulating and/or enhancing what is known as a cardiac diagnostic test/cardiopulmonary exercise test that usually requires response to an external stress (e.g., treadmill exercise) in a controlled environment. The disclosed methods mimic the increase in plasma epinephrine levels that occurs with exercise, and/or mimics changes to a subject's vital signs that occur with exercise, by administering an at least one dosage of an intranasal l-epinephrine formulation according to the disclosure.

In some embodiments, l-epinephrine is administered in conjunction with an at least one other pharmaceutical agent in the same intranasal formulation or in a separate formulation. The pharmaceutical agent may be an at least one other member of the class of adrenergic or vasoconstricting pharmaceutical agents, such as norepinephrine, vasopressin, or angiotensin II. The pharmaceutical agent may be an antihistamine, such as diphenhydramine.

In some embodiments, the transmucosal delivery of l-epinephrine as disclosed herein may be used for resuscitating a patient, e.g., for providing cardiopulmonary resuscitation and/or return of spontaneous circulation, in someone suffering from heart failure or cardiovascular collapse.

In some embodiments, a dosage of a pharmaceutical agent, such as l-epinephrine, is self-administered by a patient. In some embodiments, a dosage of the pharmaceutical agent is administered to a patient by someone other than the patient. In some embodiments, at least one dosage of the pharmaceutical agent is self-administered and at least one other dosage of the pharmaceutical agent is administered by someone other than the patient.

The present disclosure provides methods of elevating plasma epinephrine levels, which may include providing at least two dosages of one or more l-epinephrine formulations to a patient. The formulations may be adapted for oral and/or nasal inhalation and/or transmucosal, sublingual, and/or buccal absorption. The formulations may include one or more excipients and/or one or more additional active pharmaceutical ingredients. The dosages may be the same amount or different amounts. At least one of the dosages may be dispensed from a dispensing device. In some implementations, a dosage is delivered into or past either or both of the patient's nostrils for nasal inhalation or intranasal delivery. In some implementations, a dosage is delivered into or past the patient's mouth for delivery to the patient's lungs by oral inhalation, sublingual absorption, and/or buccal absorption. The two dosages may be provided at least five minutes apart. Third or subsequent dosages may be administered. Practicing the methods may maintain or substantially stabilize or normalize blood pressure, pulse, and/or breathing in the patient. Practicing the methods may transiently treat or pretreat breathing difficulty, such as to temporarily stabilize the patient or to relieve bronchoconstriction. Practicing the methods may quickly or transiently treat or pretreat breathing difficulty with an orally inhaled dosage, such that a higher dosage can then be more effectively or efficiently inhaled into or through the patient's nose for nasal inhalation or intranasal delivery. The methods may be performed when the patient is experiencing or anticipating severe allergy, anaphylaxis, anaphylactic shock, sepsis, septic shock, respiratory difficulty, and/or cardiac difficulty.

The present disclosure provides methods of elevating plasma epinephrine levels, which may include providing at least one dosage of one or more inhalable bronchodilator formulations to a patient. The inhalable bronchodilator formulations include at least one bronchodilator, which may be l-epinephrine and/or a short-acting beta-agonist such as albuterol, levalbuterol, metaproterenol, pirbuterol, or terbutaline, or a salt, derivative, or analogue thereof. In embodiments, a long-acting beta-agonist and/or corticosteroid and/or other asthma drug may additionally or alternatively be included in the formulations or methods. A dosage of albuterol, as albuterol sulfate, may include about 80 μg to about 120 μg, such as about 90 μg. Inhalable bronchodilator formulations may include at least one excipient, propellant, and/or additional active pharmaceutical ingredient. Inhalable bronchodilator formulations may be in the form of a liquid, suspension, or dry material, such as a dry powder. The inhalable bronchodilator formulations may include a propellant. The formulations may be an aerosol or be aerosolizable. The inhalable bronchodilator formulations may be adapted for oral and/or nasal inhalation and/or transmucosal absorption.

In the disclosed methods, the dosages may be dispensed from a dispensing device. The device may be an inhaler (e.g., pMDI inhaler or dry powder inhaler) or nebulizer. In some implementations, a dosage is delivered into or past either or both of the patient's nostrils for nasal inhalation or intranasal delivery. In some implementations, a dosage is delivered into or past the patient's mouth for delivery to the patient's lungs by oral inhalation.

Practicing the methods may maintain or substantially stabilize or normalize blood pressure, pulse, and/or breathing in the patient. Practicing the methods may transiently treat or pretreat breathing difficulty, such as to temporarily stabilize the patient or to relieve bronchoconstriction. In some implementations, a dosage of an inhalable bronchodilator formulation is administered by oral inhalation first, before a dosage of an inhalable l-epinephrine formulation is administered by nasal inhalation or intranasal delivery. Administration by oral inhalation first may enable a more effective or efficient subsequent inhalation into or through the patient's nose for nasal inhalation or intranasal delivery. If the bronchodilator formulation includes sulfite, and if the patient is sulfite-sensitive, subsequent administration of the inhalable l-epinephrine formulation by nasal inhalation or intranasal delivery may help to counteract any allergic reaction or hypersensitivity reaction to the sulfite/sulfate-containing bronchodilator formulation. In some implementations, a dosage of an inhalable l-epinephrine formulation is administered by nasal inhalation or intranasal delivery first, before a dosage of an inhalable bronchodilator formulation is administered by oral inhalation. If the bronchodilator formulation includes sulfite, and if the patient is sulfite-sensitive, initial administration of the inhalable l-epinephrine formulation by nasal inhalation or intranasal delivery may prevent or reduce any allergic reaction or hypersensitivity reaction to the sulfite/sulfate-containing bronchodilator formulation. In some implementations, two or more dosages of an inhalable bronchodilator formulation are administered by oral inhalation, and/or two or more dosages of an inhalable l-epinephrine formulation are administered by nasal inhalation or intranasal delivery, with or without alternating between the two types of dosages. In some implementations, an inhalable bronchodilator formulation is administered nasally.

Other variations and embodiments of the disclosure described herein will now be apparent to those of skill in the art without departing from the disclosure of the disclosure or the coverage of the claims to follow.

What is claimed is:

1. A method of elevating plasma epinephrine levels, the method comprising:
providing at least a first dosage and a second dosage of an at least one inhalable l-epinephrine formulation to a subject by dispensing at least one of said first dosage and said second dosage of the at least one inhalable l-epinephrine formulation from an at least one dispensing container into or past at least one of said subject's nostrils for nasal inhalation or intranasal delivery, and
wherein the at least one inhalable l-epinephrine formulation is adapted for transmucosal absorption, and
at least one of blood pressure, pulse, and breathing is maintained or substantially stabilized or normalized in the subject.

2. The method of claim 1, wherein the first dosage and the second dosage are provided at least five minutes apart.

3. The method of claim 1, wherein said subject is experiencing or anticipating at least one of severe allergy, anaphylaxis, anaphylactic shock, sepsis, septic shock, respiratory difficulty, and cardiac difficulty.

4. The method of claim 1, wherein the method is performed when an at least one injection of an injectable liquid l-epinephrine formulation is not available or is not possible.

5. The method of claim 1, wherein the method is performed at least until emergency medical services arrive to treat or transport said subject.

6. The method of claim 1, wherein said subject is a soldier on a battlefield or in a location without emergency medical services.

7. The method of claim 1, comprising subsequently administering an at least one injection of an injectable liquid l-epinephrine formulation.

8. The method of claim 1, comprising providing a third dosage of said at least one inhalable l-epinephrine formulation.

9. The method of claim 1, comprising alternating said subject's nostrils between dosages.

10. The method of claim 1, wherein at least one of said first dosage or said second dosage of said at least one inhalable l-epinephrine formulation comprises about 0.1 mg to about 50 mg of l-epinephrine.

11. The method of claim 1, wherein at least one of said first dosage or said second dosage of said at least one inhalable l-epinephrine formulation comprises about 1 mg to about 15 mg of l-epinephrine.

12. The method of claim 1, wherein at least one of said first dosage or said second dosage of said at least one inhalable l-epinephrine formulation comprises about 2 mg to about 8 mg of l-epinephrine.

13. The method of claim 1, wherein said dispensing into or past at least one of said subject's nostrils is by spraying into or past at least one of said subject's nostrils.

14. The method of claim 13, wherein the spraying is spraying of atomized droplets or aerosolized particles.

15. The method of claim 1, wherein at least one of said first dosage or said second dosage of said at least one inhalable l-epinephrine formulation comprises a volume of about 0.05 mL to about 0.35 mL.

16. The method of claim 1, wherein said at least one dispensing container comprises a nasal applicator having an outlet adapted to at least partially fit inside of at least one nostril of said subject.

17. The method of claim 16, wherein the nasal applicator is a nasal spray applicator or atomizing nasal spray applicator.

18. The method of claim 1, wherein said at least one dispensing container comprises a nasal spray applicator having at least one outlet adapted to at least partially fit inside of both nostrils of said subject simultaneously.

19. The method of claim 1, wherein said at least one dispensing container comprises a metered dose nasal spray applicator.

20. The method of claim 1, wherein said at least one dispensing container comprises a pressurized metered dose nasal spray applicator.

21. The method of claim 1, wherein said at least one dispensing container comprises a nasal inhaler.

22. The method of claim 1, wherein said at least one dispensing container comprises an intranasal inhaler.

23. The method of claim 1, wherein said at least one dispensing container comprises an oral inhaler.

24. The method of claim 1, wherein said at least one dispensing container comprises an inhaler adapted for both nasal inhalation and oral inhalation.

25. The method of claim 1, wherein said at least one dispensing container comprises a metered dose inhaler.

26. The method of claim 1, wherein said at least one dispensing container comprises a pressurized metered dose inhaler.

27. The method of claim 1, wherein said at least one dispensing container comprises a dry powder inhaler, dry powder sprayer, or nasal powder insufflator.

28. The method of claim 1, comprising dispensing one of said first dosage or said second dosage of the at least one inhalable l-epinephrine formulation from an at least one dispensing container into or past said subject's mouth for delivery to the subject's lungs by oral inhalation.

29. The method of claim 1, comprising dispensing one of said first dosage or said second dosage of the at least one inhalable l-epinephrine formulation from an at least one dispensing container into or past the subject's mouth for delivery by at least one of sublingual absorption or buccal absorption.

30. The method of claim 1, comprising administering a dosage of said at least one inhalable l-epinephrine formulation sublingually and/or buccally inside said subject's mouth.

31. The method of claim 1, comprising administering a dosage of said at least one inhalable l-epinephrine formulation to the subject's lungs by oral inhalation.

32. The method of claim 1, wherein at least one of said first dosage or said second dosage of said at least one inhalable l-epinephrine formulation comprises a liquid or suspension formulation.

33. The method of claim 1, wherein at least one of said first dosage or said second dosage of said at least one inhalable l-epinephrine formulation comprises a semi-viscous, gel, emulsion, or liposomal formulation.

34. The method of claim 1, wherein at least one of said first dosage or said second dosage of said at least one inhalable l-epinephrine formulation includes a propellant.

35. The method of claim 1, wherein at least one of said first dosage or said second dosage of said at least one inhalable l-epinephrine formulation comprises an aerosol or aerosolizable formulation.

36. The method of claim 1, wherein at least one of said first dosage or said second dosage of said at least one inhalable l-epinephrine formulation comprises a dry formulation.

37. The method of claim 1, wherein at least one of said first dosage or said second dosage of said at least one inhalable l-epinephrine formulation comprises a dry powder formulation.

38. The method of claim 1, wherein at least one of said first dosage or said second dosage of said at least one inhalable l-epinephrine formulation comprises a capsule or encapsulated dry powder formulation configured to open or become unencapsulated before or during inhalation.

39. The method of claim 38, wherein said capsule or encapsulated dry powder formulation is administered sublingually or buccally inside said subject's mouth.

40. The method of claim 1, wherein said first dosage and said second dosage of said at least one inhalable l-epinephrine formulation include the same amount of l-epinephrine.

41. The method of claim 1, wherein said first dosage and said second dosage of said at least one inhalable l-epinephrine formulation include different amounts of l-epinephrine.

42. The method of claim 1, wherein an amount of at least one of said first dosage or said second dosage of said at least one inhalable l-epinephrine formulation delivered to said subject is adjustable.

43. The method of claim 1, wherein the at least one inhalable l-epinephrine formulation includes at least one excipient.

44. The method of claim 1, wherein l-epinephrine is an active pharmaceutical ingredient and the at least one inhalable l-epinephrine formulation includes at least one other active pharmaceutical ingredient.

45. A method of elevating plasma epinephrine levels, the method comprising:
providing at least two dosages of an at least one inhalable l-epinephrine formulation to a subject, wherein one dosage of said at least two dosages is greater than an other dosage of said at least two dosages and is dispensed from an at least one dispensing container into or past at least one of said subject's nostrils for nasal inhalation or intranasal delivery, and
at least one of blood pressure, pulse, and breathing is maintained or substantially stabilized or normalized in the subject.

46. The method of claim 45, wherein the at least one inhalable l-epinephrine formulation is adapted for transmucosal absorption.

47. The method of claim 45, comprising dispensing said other dosage of said at least two dosages sublingually or buccally inside said subject's mouth.

48. The method of claim 45, comprising dispensing said other dosage of said at least two dosages into or past said subject's mouth for delivery to the subject's lungs by oral inhalation.

49. The method of claim 45, comprising first dispensing said other dosage of said at least two dosages into or past said subject's mouth for delivery to the subject's lungs by oral inhalation to transiently treat or pretreat breathing difficulty, temporarily stabilize the subject, or relieve bronchoconstriction.

50. The method of claim 45, comprising first dispensing said other dosage of said at least two dosages into or past said subject's mouth for delivery to the subject's lungs by oral inhalation, to quickly or transiently treat or pretreat breathing difficulty such that the one greater dosage can be more effectively or efficiently inhaled into or past at least one of said subject's nostrils compared to not first dispensing said other dosage.

51. The method of claim 45, wherein said subject is experiencing or anticipating at least one of severe allergy, anaphylaxis, anaphylactic shock, sepsis, septic shock, respiratory difficulty, and cardiac difficulty.

52. The method of claim 45, wherein the at least one inhalable l-epinephrine formulation includes at least one excipient.

53. The method of claim 45, wherein l-epinephrine is an active pharmaceutical ingredient and the at least one inhalable l-epinephrine formulation includes at least one other active pharmaceutical ingredient.

54. A method of elevating plasma epinephrine levels, the method comprising:
providing at least one dosage of an at least one inhalable bronchodilator formulation to a subject by dispensing the at least one dosage of the at least one inhalable bronchodilator formulation from an at least one dispensing container into or past the subject's mouth for delivery to the subject's lungs by oral inhalation, and
providing at least one dosage of an at least one inhalable l-epinephrine formulation to said subject by dispensing said at least one dosage of the at least one inhalable l-epinephrine formulation from an at least one dispensing container into or past at least one of said subject's nostrils for nasal inhalation or intranasal delivery, and
wherein the at least one inhalable l-epinephrine formulation is adapted for transmucosal absorption, and
at least one of blood pressure, pulse, and breathing is maintained or substantially stabilized or normalized in the subject.

55. The method of claim 54, wherein l-epinephrine is a bronchodilator and the at least one inhalable bronchodilator formulation includes at least one other bronchodilator.
